(19) United States
(12) Patent Application Publication
(10) Pub. No.: US 2011/0293593 A1
(43) Pub. Date: Dec. 1, 2011

(54) SIVA 2 STABILIZATION

(75) Inventors: David Wallach, Rehovot (IL); Parameswaran Ramakrishnan, Trivandrum (IN); Andrei Kovalenko, Rehovot (IL)

(73) Assignee: YEDA RESEARCH AND DEVELOPMENT CO., LTD., Rehovot, IL (IL)

(21) Appl. No.: 12/866,532

(22) PCT Filed: Feb. 11, 2009
(86) PCT No.: PCT/IL2009/000161

§ 371 Date: May 12, 2011

(30) Foreign Application Priority Data
Feb. 10, 2008 (IL) 189405

(51) Int. Cl. A61K 38/45; C12P 21/00; C12N 5/10; C12N 1/19; A61P 29/00; C07K 1/107; C07K 19/00; G01N 33/566; C12Q 1/48; A61P 35/00; C07K 14/47; A61K 38/17

(52) U.S. Cl. 424/94.5; 530/352; 530/395; 530/350; 435/69.1; 435/325; 435/348; 435/367; 435/369; 435/358; 435/254.2; 514/20.9; 514/1.1; 435/68.1; 530/409; 436/501; 435/15; 514/19.3

(57) ABSTRACT

The present invention relates to modulation of SIVA2 stability by N-acetylglucosamine, phosphorylation of ubiquitination in treatment or prevention of diseases, disorders or conditions.

### FIELD OF THE INVENTION

The present invention relates to modulation of SIVA2 stability in treatment or prevention of diseases, disorders or conditions.

### BACKGROUND OF THE INVENTION

Members of the TNF/NGF receptor family are expressed in almost all types of cells and control a wide range of diverse cellular activities. They have the ability both to induce cellular changes that are protein-synthesis independent, the best known of which is caspase-mediated cell death (the extrinsic cell-death pathway), and to modulate gene-expression patterns both on the transcriptional and the post-transcriptional levels. These effects contribute to the control of practically all aspects of immune defense as well as some embryonic-development and tissue-homeostatic processes. They vary, and depending on the type of cell and the identity of the activated receptor, as well as on numerous other determinants, some effects might even oppose others. This wide range of activities is mediated by a rather small number of signaling proteins, of which the best characterized are two death-domain-containing adapters, FADD/MORT1 and TRADD, the inducer caspases caspase-8 and -10, members of the TRAF ring-finger proteins, and cellular inhibitor of apoptosis protein 1 (cIAP1) and cIAP2 (ring-finger proteins with IAP motifs) (Wallach et al., 1999)'(Locksley et al., 2001). How this limited set of proteins mediates the multiplicity of different effects of the receptors, and how the nature of the induced effect is adjusted to need, are still poorly understood.

SIVA, an additional protein suggested to participate in the proximal signaling activities of members of the TNF/NGF receptor family, was identified by virtue of its binding to the receptor CD27 in the yeast two-hybrid test (Prasad et al., 1997). Some evidence was also presented for its association with several other members of the TNF/NGF receptor family (Nocentini and Riccardi, 2005). The existence of SIVA has been known for some years, and it was shown that when overexpressed for prolonged periods this protein kills cells (Prasad et al., 1997). However, whether this is its genuine and sole activity is not known. SIVA shows no close structural resemblance to any other known protein. One region within it that initially appeared to resemble the death domain does not contain the structural signatures by which that domain is characterized. C-terminally to that region the protein is relatively enriched in cysteine residues, which apparently contribute to its binding of several zinc ions (Nestler et al., 2006). The amino-acid sequence in this region, however, does not strictly conform to any of the known zinc-binding motifs. A central short α-helical region in the protein binds the anti-apoptotic protein BCL-X_{L} (Xue et al., 2002), but the function served by the cysteine-rich region (CRR) is unknown.

SIVA it is known to exist as two alternative splice isoforms or splice variants, SIVA1 and SIVA2. SIVA1 is longer and contains a death domain homology region (DDHR) with a putative amphipathical helix in its central part. SIVA2 is shorter and lacks the DDHR. Both isoforms contain a B-box-like ring finger and a Zinc finger like domain in their C-termini. Enforced expression of both SIVA1 and SIVA2 has been shown to induce apoptosis (Prasad et al., 1997, Yoon et al., 1998, Spinicelli et al., 2003, (Py et al., 2004). SIVA1 induced apoptosis is suggested to be effected by its binding to and inhibition of the anti apoptotic Bcl-2 family members through its amphipathic helical region (Chu et al., 2005; Chu et al., 2004; Xue et al., 2002). Consistent with its pro-apoptotic role, SIVA is a direct transcriptional target for tumor suppressors p53 and E2F1 (Fortin et al., 2004). Various point of evidence indicate that SIVA is a stress-induced protein and is up-regulated in acute ischemic injury (Padanilam et al., 1998), coxavirus infection (Henke et al., 2000), and also by cisplatin treatment (Qin et al., 2002), as well as TIP30 expression which induces apoptosis (Xiao et al., 2000). Recently, the common N- and C-termini of SIVA1 and SIVA2, yet not the death domain, have been shown to be sufficient and capable to mediate apoptosis in lymphoid cells through activation of a caspase dependent mitochondrial pathway (Py et al., 2004).

Recently, it was found that SIVA binds to NF-κB-inducing kinase (NIK) and controls its function (Ramakrishnan et al., 2004), has ubiquitination-related activity, is capable of directly inducing self-ubiquitination, ubiquitination of TRAF2 (a TNF-receptor associated adaptor protein 2), and that SIVA2 is an E3 ligase (WO2007080593).

Ubiquitylation, also termed ubiquitination, refers to the process particular to eukaryotes whereby a protein is post-translationally modified by covalent attachment of a small protein named ubiquitin [originally ubiquitous immunopoeitic polypeptide (UBIP)]. Ubiquitin ligase is a protein which covalently attaches ubiquitin to a lysine residue on a target protein. The ubiquitin ligase is typically involved in polyubiquitylation: a second ubiquitin is attached to the first; a third is attached to the second, and so forth. The ubiquitin ligase is referred to as an “E3” and operates in conjunction with an ubiquitin-activating enzyme (referred herein as “E1”) and an ubiquitin-conjugating enzyme (referred herein as “E2”). There is one major E1 enzyme, shared by all ubiquitin ligases, which uses ATP to activate ubiquitin for conjugation and transfers it to an E2 enzyme. The E2 enzyme interacts with a specific E3 partner and transfers the ubiquitin to the target protein. The E3, which may be a multi-protein complex, is generally responsible for targeting ubiquitination to specific substrate proteins. In some cases it receives the ubiquitin from the E2 enzyme and transfers it to the target protein or substrate protein; in other cases it acts by interacting with both the E2 enzyme and the substrate.

NIK, (MAP3K14) was discovered (Malinin et al., 1997) in a screening for proteins that bind to TRAF2. The marked activation of NF-κB upon overexpression of NIK, and effective inhibition of NF-κB activation in response to a variety of inducing agents, upon expression of catalytically inactive NIK mutants suggested that NIK participates in signaling for NF-κB activation (Malinin et al., 1997).

Assessment of the pattern of the NF-κB species in lymphoid organs indicated that, apart from its role in the regulation of NF-κB complex(s) comprised of Rel proteins and IκB, NIK also participates in controlling the expression/activation of other NF-κB species. Indeed, NIK has been shown to participate in site-specific phosphorylation of p100, which serves as a molecular trigger for ubiquitination and active processing of p100 to form p52. This p100 processing activity was found to be ablated by the aly mutation of NIK (Xiao et al., 2001b).

NIK in thymic stroma is important for the normal production of Treg cells, which are essential for maintaining immunological tolerance. NIK mutation resulted in disorganized thymic structure and impaired production of Treg cells in aly mice (Kajiura et al., 2004). Consistently, studies of NIK-deficient mice also suggested a role for NIK in controlling the development and expansion of Treg cells (Lu et al., 2005). These findings suggest an essential role of NIK in establishing self-tolerance in a stromal dependent manner. NIK also partakes in NF-κB activation as a consequence of viral infection. Respiratory syncytial virus infection results in increased kinase activity of NIK and the formation of a complex comprised of activated NIK, IKK1, p100 and the processed p52 in alveolus like a549 cells. In this case NIK itself gets translocated into the nucleus bound to p52 and surprisingly, these events precede the activation of canonical NF-κB pathway activation (Choudhary et al., 2005). These findings indicate that NIK indeed serves as a mediator of NF-κB activation, but may also serve other functions, and that it exerts these functions in a cell- and receptor-specific manner.

NIK can be activated as a consequence of phosphorylation of the ‘activation loop’ within the NIK molecule. Indeed, mutation of a phosphorylation-site within this loop (Thr-559) prevents activation of NF-κB upon NIK overexpression (Lin et al., 1999). In addition, the activity of NIK seems to be regulated through the ability of the regions upstream and downstream of its kinase motif to bind to each other. The C terminal region of NIK downstream of its kinase moiety has been shown to be capable of binding directly to IKK1 (Regnier et al., 1997) as well as to p100 (Xiao et al., 2001b) and these interactions are apparently required for NIK function in NF-κB signaling. The N terminal region of NIK contains a negative-regulatory domain (NRD), which is composed of a basic motif (BR) and a proline-rich repeat motif (PRR) (Xiao and Sun, 2000). The N-terminal NRD interacts with the C-terminal region of NIK in cis, thereby inhibiting the binding of NIK to its substrate (IKK1 and p100). Ectopically expressed NIK spontaneously forms oligomers in which these bindings of the N-terminal to the C terminal regions in each NIK molecule are apparently disrupted, and display a high level of constitutive activity (Lin et al., 1999). The binding of the NIK C-terminal region to TRAF2 (as well as to other TRAF's) most likely participates in the activation process. However, its exact mode of participation is unknown.

Recently, a novel mechanism of NIK regulation has gained much attention. This concerns the dynamic interaction of NIK and TRAF3 leading to proteasome mediated degradation of NIK. Interestingly, inducers of the alternative pathway of NF-κB like CD40 and BLyS have been shown to induce TRAF3 degradation and concomitant enhancement of NIK expression (Liao et al., 2004).

There is rather limited information yet of the downstream mechanisms in NIK action. Evidence has been presented that NIK, through the binding of its C-terminal region to IKK1 can activate the IκB kinase (IKK) complex. It has indeed been shown to be capable of phosphorylating serine-176 in the activation loop of IKK1 and thereby its activation (Ling et al., 1998).

It was suggested that NIK does not participate at all in the canonical NF-κB pathway, but rather serves exclusively to activate the alternative one (see (Pomerantz and Baltimore, 2002, for review). However, it was lately shown that although the induction of IkappaB degradation in lymphocytes by TNF is independent of NIK, its induction by CD70, CD40 ligand, and BLyS/BAFF, which all also induce NF-kappaB2/p100 processing, does depend on NIK function (Ramakrishnan et al. 2004). Both CD70 and TNF induce recruitment of the IKK kinase complex to their receptors. In the case of CD70, but not TNF, this process is associated with NIK recruitment and is followed by prolonged receptor association of just IKK1 and NIK. Recruitment of the IKK complex to CD27, but not that of NIK, depends on NIK kinase function. These findings indicate that NIK participates in a unique set of proximal signaling events initiated by specific inducers, which activate both canonical and noncanonical NF-kappaB dimers.

Yamamoto and Gaynor reviewed the role of NF-κB in pathogenesis of human disease (Yamamoto and Gaynor 2001). Activation of the NF-κB pathway is involved in the pathogenesis of chronic inflammatory disease, such as asthma, rheumatoid arthritis (see Tak and Firestein, this Perspective series, ref. Karin et al. 2000), and inflammatory bowel disease. In addition, altered NF-κB regulation may be involved in other diseases such as atherosclerosis (see Collins and Cybulsky, this series, ref. Leonard et al. 1995) and Alzheimer's disease (see Mattson and Camandola, this series, ref. Lin et al. 1999), in which the inflammatory response is at least partially involved. Also, abnormalities in the NF-κB pathway are also frequently seen in a variety of human cancers.

Several lines of evidence suggest that NF-κB activation of cytokine genes is an important contributor to the pathogenesis of asthma, which is characterized by the infiltration of inflammatory cells and the deregulation of many cytokines and chemokines in the lung (Ling et al. 1998). Likewise, activation of the NF-κB pathway also likely plays a role in the pathogenesis of rheumatoid arthritis. Cytokines, such as TNF-, that activate NF-κB are elevated in the synovial fluid of patients with rheumatoid arthritis and contribute to the chronic inflammatory changes and synovial hyperplasia seen in the joints of these patients (Malinin et al. 1997). The administration of antibodies directed against TNF- or a truncated TNF-receptor that binds to TNF—can markedly improve the symptoms of patients with rheumatoid arthritis.

Increases in the production of proinflammatory cytokines by both lymphocytes and macrophages have also been implicated in the pathogenesis of inflammatory bowel diseases, including Crohn's disease and ulcerative colitis (Matsumoto et al. 1999). NF-κB activation is seen in mucosal biopsy specimens from patients with active Crohn's disease and ulcerative colitis. Treatment of patients with inflammatory bowel diseases with steroids decreases NF-κB activity in biopsy specimens and reduces clinical symptoms. These results suggest that stimulation of the NF-κB pathway may be involved in the enhanced inflammatory response associated with these diseases.

Atherosclerosis is triggered by numerous insults to the endothelium and smooth muscle of the damaged vessel wall (Matsushima et al. 2001). A large number of growth factors, cytokines, and chemokines released from endothelial cells, smooth muscle, macrophages, and lymphocytes are involved in this chronic inflammatory and fibroproliferative process (Matsushima et al. 2001). NF-κB regulation of genes involved in the inflammatory response and in the control of cellular proliferation likely plays an important role in the initiation and progression of atherosclerosis.

Also, abnormalities in the regulation of the NF-κB pathway may be involved in the pathogenesis of Alzheimer's disease. For example, NF-κB immunoreactivity is found predominantly in and around early neuritic plaque types in Alzheimer's disease, whereas mature plaque types show vastly reduced NF-κB activity (Mercurio et al. 1999). Thus, NF-κB activation may be involved in the initiation of neuritic plaques and neuronal apoptosis during the early phases of Alzheimer's disease. These data suggest that activation of the NF-κB pathway may play a role in a number of diseases that have an inflammatory component involved in their pathogenesis.

In addition to a role in the pathogenesis of diseases characterized by increases in the host immune and inflammatory response, constitutive activation of the NF-κB pathway has also been implicated in the pathogenesis of some human cancers. Abnormalities in the regulation of the NF-κB pathway are frequently seen in a variety of human malignancies including leukemias, lymphomas, and solid tumors (Miyawaki et al. 1994). These abnormalities result in constitutively high levels of NF-κB in the nucleus of a variety of tumors including breast, ovarian, prostate, and colon cancers. The majority of these changes are likely due to alterations in regulatory proteins that activate signaling pathways that lead to activation of the NF-κB pathway. However, mutations that inactivate the I B proteins in addition to amplification and rearrangements of genes encoding NF-κB family members can result in the enhanced nuclear levels of NF-κB seen in some tumors.

Apart from the contribution to the regulation of the development and function of the immune system, NIK seems also to be involved in the regulation of various non-immune functions such as mammary gland development (Miyawaki et al., 1994). NIK has a role in lymphoid organ development (Shinkura et al., 1999). In vitro studies implicated NIK in signaling that leads to skeletal muscle cell differentiation (Canicio et al., 2001), and in the survival and differentiation of neurons (Foehr et al., 2000).

A need of a satisfactory treatment exists for numerous lethal and/or highly debilitating diseases associated with disregulated activity of SIVA, NIK and/or NF-.κ.B molecules, including malignant diseases and diseases associated with pathological immune responses, such as autoimmune, allergic, inflammatory, and transplantation-related diseases.

### SUMMARY OF THE INVENTION

In one aspect, the invention provides a stability-improved SIVA2 or salt thereof characterized by comprising the following post translation modification(s) (i) O-GlcNAcylation; (ii) phosphorylation at serine residues 5, 50, and 51 (iii) ubiquitination on residues, K17 and/or K99; or (iv) a combination of (i) to (iii).

In one embodiment of the invention, the stability-improved SIVA2 is also phosphorylated at serine residues 21, 26, and 35.

In another aspect, the invention provides a method of preparing a stability-improved SIVA2 characterized by comprising the following post translation modification(s) (i) O-GlcNAcylation; (ii) phosphorylation at serine residues 5, 50, 51 of SIVA2; (iii) ubiquitination on SIVA2 residues, K17 and/or K99; or (iv) a combination of (i) to (iii), the method comprising over-expressing in an eukaryotic cell recombinant or endogenous SIVA2 and increasing in said cell the levels of (a) TRAF2, (b) a ring-finger mutant of cIAP1, (c) a O-GlcNAc transferase, (d) an inhibitor of O-GlcNAcase, (e) UDP-GlcNac (f) a combination of (a) to (e) or (g) increasing the levels of NIK and any one of (a) to (f).

In one embodiment of the invention, it is provided a method of preparing stability-improved SIVA2 that is carried out ex-vivo, and includes culturing said cell under conditions allowing production of said stability-improved SIVA2 and recovering the resulting stability-improved SIVA2 from the culture. Also, it is provided according to the invention a host cell comprising a stability-improved SIVA2 and an isolated stability-improved SIVA2 prepared according to the method of the invention.

In a further aspect, the invention provides a pharmaceutical composition comprising a pharmaceutically acceptable carrier and a stability-improved SIVA2 or salt thereof characterized by comprising one or more of the following post translation modification(s) (i) O-GlcNAcylation; (ii) phosphorylation at serine residues 5, 50, and 51; (iii) ubiquitination on residues, K17 and/or K99; or (iv) a combination of (i) to (iii). In one embodiment of the invention, the pharmaceutical composition can be used for treating a disease, disorder, or condition associated with low activity or level of SIVA2 or ameliorated by increasing the activity or level of a SIVA2 in cells; and/or for treating a disease, disorder or condition in which a signaling pathway activated by a member of the TNF/NGF receptor family is associated with the pathogenesis or course said disease, disorder or condition for example, cancer, inflammatory diseases, and/or autoimmune diseases.

It is one aim of the invention to provide the use of a stability-improved SIVA2 characterized by comprising one or more of the following post translation modification(s) (i) O-GlcNAcylation; (ii) serine phosphorylation at serine residues 5, 50, and 51; or (iii) ubiquitination on residues, K17 and/or K99, for treating, or in the manufacture of a medicament for treating a disease, disorder, or condition associated with low activity or level of SIVA2 or ameliorated by increasing the activity or level of a SIVA2 in cells; and/or treating a disease, disorder or condition in which signaling of a pathway mediated by a member of the TNF/NGF receptor family is associated with the pathogenesis or course said disease disorder or condition such as cancer, inflammatory diseases, and/or an autoimmune diseases.

It is another aim of the invention to provide a method for stabilizing SIVA2 comprising contacting SIVA2 with an O-GlcNac transferase, TRAF2, an inhibitor of O-GlcNAcase, an inhibitor CIAP1 activity, a ring-finger mutant of cIAP1 such as H588A or a combination thereof. Said contacting can be carried out in vivo, in vitro or ex-vivo.

It is a further aim of the invention to provide the use of an agent capable of altering SIVA2 stability selected form (i) an agent capable of modulating O-GlcNacidation, (ii) an agent capable of modulating TRAF2 activity, (iii) an agent capable of modulatring CIAP1 activity, and/or (iv) a ring-finger mutant of cIAP1 such as H588A for treating of a disease, disorder, or condition in which a signaling pathway by a member of the TNF/NGF receptor family is associated with the pathogenesis or course of the disease, disorder, or condition.

In one embodiment of the invention, altering SIVA2 stability consists on improving SIVA2 stability and the agent which can be used to improve SIVA2 stability is for example, O-GlcNac transferase, an inducer of O-GlcNac transferase such as UDP-GlcNac, TRAF2, an inhibitor of O-GlcNAcase, an inhibitor CIAP1 activity, a ring-finger mutant of cIAP1 such as H588A or a combination thereof. Improving SIVA2 stability can be used for treating cancer, an inflammatory disease, and/or an autoimmune disease.

In another embodiment of the invention, altering SIVA2 stability consists on diminishing or reducing SIVA2 stability and the agent which can be used is an inhibitor of O-GlcNac transferase, inhibitor of TRAF2, O-GlcNAcase, CIAP1, or a combination thereof. Diminishing or reducing SIVA2 stability can be used for treating an immune deficiency or ischemia/reperfusion.

In another aspect, the invention provides a complex of SIVA2 or stability-improved SIVA2 with cIAP. In a further embodiment of the invention, it is provided a complex of SIVA2 with cIAP1.

In a further aspect, the invention provides a method for screening a molecule capable of modulating signaling by a member of the TNF/NGF receptor family in a disease disorder or condition comprising contacting SIVA2 with cIAP and/or TRAF2, monitoring the level of the complex of SIVA2 with cIAP and/or TRAF2 in the presence and in the absence of a candidate molecule, wherein a change in the level of SIVA2-cIAP and/or SIVA2-TRAF2 complex in the presence of the candidate molecule is indicative that the candidate molecule modulates signaling by said member of the TNF/NGF family.

In one embodiment of the invention, the method is for screening a molecule capable of downregulating signaling by the member of the TNF/NGF receptor family in a disease disorder or condition such as an automimmune disease, disorder or condition or in kidney ischemia and wherein the candidate molecule increases the level of the complex.

In another embodiment of the invention, the method is for screening a molecule capable of prolonging signaling by the member the TNF/NGF receptor family in a disease, disorder or condition such as a condition associated with immunosuppression and wherein the candidate molecule decreases the level of the complex.

In a still further aspect of the invention, it is provided a method for screening a molecule capable of modulating signaling by a member of the TNF/NGF receptor family in a disease, disorder or condition comprising inducing SIVA2 stability in the presence and in the absence of a candidate molecule, wherein a change in the level of stability-induced SIVA2 in the presence of a candidate molecule is indicative that the candidate molecule can modulate signaling by the member of the TNF/NGF receptor family.

In one embodiment of the invention, the method is for screening a molecule capable of downregulating signaling by the member of the TNF/NGF receptor family in a disease, disorder or condition such as in automimmune disease, disorder or condition or in kidney ischemia and wherein the candidate molecule increases the level of stabilized SIVA2.

In another embodiment, the method is for screening a molecule capable of prolonging signaling by the member of the TNF/NGF receptor family in a disease, disorder or condition, for example, associated with immunopsuppression and wherein the candidate molecule decreases the level of stabilized SIVA2.

The invention also provides, a method for treating a disease, disorder, or condition in which a signaling pathway by a member of the TNF/NGF receptor family is associated with the pathogenesis or course of the disease, disorder, or condition wherein the method comprises administration of a therapeutically effective amount an agent capable of altering SIVA2 stability selected from (i) an agent capable of modulating O-GlcNacidation, (ii) an agent capable of modulatring TRAF2 activity, (iii) an agent capable of modulatring CIAP1 activity, (iv) a ring-finger mutant of cIAP1 such as H588A.

### BRIEF DESCRIPTION OF THE FIGURES

FIG. 1 shows that SIVA2 is stabilized by several ligands of the TNF family. (A) Expression of SIVA1 and SIVA2 in various cell lines. For each cell line, cellular protein (30 μg) was resolved by 13.5% SDS-PAGE and probed with anti-SIVA antibody. The two lanes at the right show SIVA1 and SIVA2 overexpressed in HEK-293T cells (for each protein, 2 μg cDNA/well in 6-well plates). (B) Several SIVA isoforms are expressed in PBMCs. RT-PCR shows expression of SIVA1, SIVA2, and SIVA3 in resting PBMCs. (C) Ligand activation increases the amount of SIVA2 in resting PBMCs. Cells (2×10⁶) were treated with the indicated ligands for 8 h and the cell lysates were analyzed by western blotting. (D) Ligand activation and proteasomal inhibition, but not genotoxic stress, increase SIVA2 levels in activated PBMCs. The cells were stimulated with PHA (1 μg/ml) for 48 h, washed twice with phosphate-buffered saline, and incubated for an additional period of 18 h without PHA. Ligands and genotoxic agents (camptothecin (CPT) 10 μM and cisplatin (CIS) 50 μM) were then applied for 18 h. MG132 was applied for the last 4 h of treatment. Except where otherwise indicated, MG132 was applied in this study at a concentration of 25 ‘ns’ denotes a non-specific band serving as loading control. (E) SIVA2 message does not increase after ligand activation. PBMCs were activated as in FIG. 1d. Ligands were applied to the indicated cell types for 18 h. Semi-quantitative RT-PCR for SIVA message was performed as described in Methods. GAPDH was used as a basis for normalization. (F) Stabilization of transiently expressed SIVA2 by ligands of the TNF family in EcR-293-CD27 and EcR-293-CD40 cells. SIVA2 or SIVA1 plasmids (0.5 μg) were transfected, and 18 h later ligands were applied for 8 h. Total-cell lysates were analyzed by western blotting using anti-SIVA antibody. TNF-induced SIVA2 stabilization was assessed in EcR-293-CD27 cells. (G) Inducibly expressed SIVA2 is stabilized by CD70. EcR-293-CD27-SIVA2 cells (see Methods) were treated with ponasterone and CD70 as indicated. Total-cell lysates were analyzed by western blotting using LDH as the loading control (bottom panel). (H) Proteasomal inhibition stabilizes transiently expressed SIVA2 and enhances accumulation of the polyubiquitinated protein. HEK-293T cells were transfected with FLAG-SIVA2 and analyzed 24 h after transfection. MG132 was applied for the last 4 h of treatment. TCL, total cell lysate. (I) Differential effects of genotoxic agents on the expression of SIVA1 and SIVA2. Top: HepG2 cells were treated with CPT for 18 h. When indicated, they were transfected with pSUPER SIVA 30 h prior to CPT application. Cells lysates were analyzed by western blotting using anti-SIVA antibody. Middle: HepG2 cells were exposed to UVC (20 J/m²) and levels of SIVA proteins were determined after 18 h of treatment. Last lane (‘Control’) in the top and middle panels shows SIVA2 overexpressed with NIK and endogenous SIVA1 in HEK-293T cells. Bottom: HEK-293T cells were transfected with 0.5 μg of FLAG-SIVA2. CPT and CD40L were applied, 8 h after transfection, for a further 18 h.

FIG. 2 shows that TRAF2 and NIK, independently, contribute to ligand-induced stabilization of SIVA2 while cIAP1 facilitates its degradation (A) NIK, but not enzymatically inactive NIK, stabilizes SIVA. SIVA2 was cotransfected with wild-type or enzymatically inactive NIK mutant, KD-NIK, in HEK-293T cells, and lysates were analyzed for SIVA and NIK expression 24 h after transfection. (B) TRAF2 stabilizes SIVA2 independently of NIK. The plasmids were transfected into HEK-293T cells as indicated, and lysates were analyzed 24 h after transfection. (C, D) Both NIK and TRAF2 are essential for CD70-induced SIVA2 stabilization. (C), Plasmids were transfected into EcR-293-CD27 cells 24 h after transfection of TRAF2 siRNA. CD70 was applied for the last 18 h of the 48-h period of treatment starting from the time of the first transfection. (D) SIVA2 and KD-NIK were transiently cotransfected into EcR-293-CD27 cells and treated with CD70 for the last 18 h of the 28-h transfection. (E) Both NIK and TRAF2 are essential for CD40-induced SIVA2 stabilization. EcR-293-CD40 cells were transfected with the indicated plasmids, and after 8 h CD40L was applied for 18 h. Total plasmid concentration in the transfection was maintained by the use of empty vectors. Green fluorescent protein (GFP) plasmid was used to monitor transfection uniformity. (F) TRAF2, but not NIK contribute to TNF-induced SIVA2 stabilization. HEK-293T cells were transfected with SIVA2 and pSUPER NIK or TRAF2 siRNA, as described above. TNF was applied at the indicated times before the cells were harvested. (G) NIK stabilizes SIVA2 independently of TRAF2. The plasmids were transfected into HEK-293T cells 24 h after transfection of TRAF2 siRNA. Lysates were prepared 48 h after the first transfection and analyzed for SIVA2 and TRAF2. (H) Effect of cIAP1 and its H588A mutant on SIVA2 expression. HEK-293T cells were seeded in 6 well plates and co-transfected with FLAG-SIVA2 and FLAG cIAP1 or FLAG cIAP1 (H588A) plasmids. 28 h post transfection, the cells were harvested and SIVA2 and cIAP1 levels were assessed by western blotting. The arrow points to a modified form of SIVA2 that accumulates in cells transfected with cIAP1 (H588A).

FIG. 3 shows that SIVA is O-linked N-acetylglucosamine modified and this kind of modification contributes to its stabilization by TRAF2 and NIK.(A) SIVA2 incorporates azido-GlcNAc in cells. HEK-293T cells cotransfected with NIK and SIVA2 were metabolically labeled with azido-GlcNAc, in-vitro biotinylated, and immunoprecipitated. The biotin-labeled GlcNAc moieties in SIVA2 were detected with streptavidin horseradish peroxidase (HRP). (B) SIVA binds to wheatgerm-agglutinin. The plasmids for SIVA2, alone or together with NIK or TRAF2, were transiently expressed in HEK-293T cells. Lactacystin was applied for the last 6 h of the 24-h treatment period. Lysates were analyzed for expression (right panel) and WGA binding (left panel) of SIVA2. N-acetyl-D-glucosamine (0.5 M) was added as a competitor for WGA binding. (C) β-D-N-acetyl hexosaminidase treatment abolishes binding of SIVA2 to WGA. FLAG-SIVA2 was cotransfected with myc-NIK into HEK-293T cells and immunoprecipitated with anti-FLAG-M2 beads. The immunoprecipitated beads were boiled with 1% SDS and the eluted proteins were treated with ii-D-N-acetyl hexosaminidase as described (Whelan, 2006). The samples were collected after treatment for 4, 8, and 20 h, diluted with WGA binding buffer, and lectin binding was assayed as described in Methods. Immunoprecipitation of the protein with anti-FLAG-M2 beads after treatment for 8 h as described above, followed by western analysis, confirmed that despite having lost the ability to bind to WGA the protein remained intact. ‘Sup’, SIVA protein remaining unbound after the reaction. (D) Inhibition of O-glycosylation interferes with TRAF2-induced SIVA2 stabilization, but does not affect MG132-induced stabilization. The plasmids were transiently expressed in HEK-293T cells. DON (50 μM) was applied for the last 16 h and MG132 for the last 6 h of the 28-h treatment period. (E) Inhibition of O-glycosylation blocks the NIK-mediated stabilization of wild-type SIVA2, but has no effect on the residual stabilization by NIK of the SIVA2 6SA mutant. The experiment was performed as in (D). (F) Specific inhibition of O-GlcNAcylation blocks NIK-induced SIVA2 stabilization. HEK-293T cells were cotransfected with NIK and SIVA2 and then treated with 0.7, 1.4, or 2.0 mM BADGP for the last 16 h of a 28-h treatment. The first lane shows expression of SIVA2 in cells treated only with the BADGP solvent.

FIG. 4 shows that SIVA2 is phosphorylated in mutiple serine residues at its N-terminus and this phosphorylation as well seems to contribute to its stabilization. (A) SIVA2 is phosphorylated in cells. HEK-293T cells transiently expressing myc-NIK and FLAG-SIVA2 were metabolically labeled with [³²P]orthophosphate. MG132 was applied for the last 6 h of treatment. (B) Assignment of the phosphorylation sites in SIVA2 isolated from cells overexpressing NIK. The peptides comprising phosphorylated residues of SIVA2 were located by precursor ion scan in a negative ion mode at m/z −79. Phosphorylated residues were later assigned by tandem nano-electrospray MS analysis in a positive mode (see Table SI). Shown at the top is the amino-acid sequence of SIVA2 with a schematic presentation of very likely (in bold) and confirmed (in bold and designated pSⁿ) phosphorylated residues in SIVA2. (C) SIVA2 in vitro phosphorylation. Effect of serine mutations. myc-NIK and FLAG-SIVA2 and its indicated serine mutants (3SA, replacement of residues 5, 50 and 51 by alanines, and 6SA, replacement of residues 5, 21, 26, 35, 50 and 51 by alanines) were cotransfected into HEK-293T cells and the immunoprecipitated SIVA was subjected to an in-vitro kinase assay{Ramakrishnan, 2004}. Bottom panel shows normalized total amounts of SIVA2 and its mutants in the kinase reaction. Western blot analysis of the coprecipitated NIK confirmed that its amount in the precipitate was not decreased by the 3SA or the 6SA mutations. (D) NIK expression or proteasomal inhibition stabilizes SIVA N-terminus. HEK-293T cells were transfected with FLAG-SIVA2 (1-58) and myc-NIK as indicated. MG132 (25 uM) was applied for the last 6 hours of 24 h transfection. Cells were harvested, lysed and SIVA2 levels were assessed by anti-FLAG antibody. (E) NIK co-expression enhances phosphorylation of SIVA2 (1-58). Phosphorylation of SIVA2 (1-58) in cells was assessed by metabolic labeling with [³²P]orthophosphate, 22 h after transfection of the indicated plasmids. Okadaic acid (1 μM) was added for the last 45 min.

FIG. 5 shows identification of amino acid residues in SIVA2 that contribute to its stabilization by NIK and TRAF2. (A) Individual serine mutations do not interfere with NIK-induced SIVA2 stabilization. Different serine-mutant SIVA2 plasmids were cotransfected with NIK into HEK-293T cells and the cell lysates were analyzed 24 h after transfection. (B) Tyrosine 34 of SIVA2 does not participate in its phosphorylation or stabilization by NIK. The indicated plasmids were transfected into HEK-293T cells, and 24 h later SIVA2 and NIK in the lysates were determined (top two panels). Bottom panel: phosphorylated SIVA2 from an in-vitro kinase assay, performed as in FIG. 4C, with the SIVA and SIVA-mutant proteins immunoprecipitated from cells co-expressing NIK.(C) Combined mutation of several of residues in SIVA2 that can be phosphorylated interfere with the protein's NIK-induced stabilization. Each of the indicated plasmids was transfected into HEK-293T cells, and the amount of SIVA2 and NIK in cell lysates was determined 24 h after transfection. (D) TRAF2 and proteasomal inhibition stabilize the SIVA2 serine mutants that cannot be stabilized by NIK. Plasmids were transfected as described above. MG132 was added 18 h later, and after a further 6 h the cellular proteins were extracted. (E) Combined serine mutation in SIVA2 compromises its stabilization by CD40L. EcR-293-CD40 cells were transfected with 0.75 μg of the SIVA2 plasmid or with 1.5 μg of the SIVA2 6SA mutant plasmid. CD40L was applied at the indicated times before cell harvesting, which was carried out 30 h after transfection. (F) Lysines in SIVA2 participate in its stabilization by TRAF2. The indicated plasmids were cotransfected into HEK-293T cells. Total-cell lysates were prepared 24 h after transfection and analyzed by western blotting. (G) The lysines contributing to SIVA2 stabilization by TRAF2 are not involved in its stabilization by NIK. SIVA2 and NIK expression levels were determined as above.

FIG. 6 SIVA2 is recruited to receptors of the TNF/NGF family and binds specifically to NIK, TRAF2, and cIAP1. (A) Transfected SIVA2 binds to endogenous TRAF2. FLAG-SIVA2 or HIS-SIVA2 (control) was transfected into HEK293T cells. SIVA2 was immunoprecipitated using anti-FLAG M2 beads and the co-precipitated cellular TRAF2 was assayed by western blotting. The total cellular level of TRAF2 is shown at the bottom. (B) SIVA2 binds TRAF2 inducibly. Treatment of the cells EcR293-CD27-SIVA2 with ponasterone for 2 h to induce SIVA2, and treated with CD70 as indicated was followed by immunoprecipitation of TRAF2. (C) SIVA2 binds TRAF2 in vitro. FLAG-tagged TRAF2 was immunoprecipitated from transfected HEK293T cells with anti-FLAG M2 beads, eluted from the beads using FLAG peptide and incubated with GST-SIVA2 or its mutant, and then subjected to immunoprecipitation and western blotting as indicated. (D) SIVA2 binds at its N-terminus to cIAP1. Left panel: Binding in vitro. Recombinant cIAP1 was incubated with GST-SIVA2 or its mutant. Right and bottom panels depict binding in transfected HEK293T cells. Right panel: Cells were transfected with HIS-SIVA2, HIS-SIVA2 (1-58), or FLAG-TRAF2. After 28 h the endogenous cIAP1 was immunoprecipitated. MG132 was applied for the last 6 h of incubation. Bottom panel: Cells were transfected with FLAG-SIVA2, FLAG-SIVA2 (1-58) or, as a specificity control, FLAG-GST-BR3-ICD* (in which the BAFF receptor intracellular domain is mutated at its TRAF3-binding region (PVPAT>AVAAA)). After 28 h the transfected proteins were immunoprecipitated and probed for co-precipitated endogenous cIAP1. MG132 was applied for the last 6 h of incubation. (E) Diagrammatic representation of the deletion analyses of SIVA2 binding to cIAP1, NIK, and TRAF2 presented in FIGS. 6D and H. Left: the deletion mutants used. Right: the binding observed. N/A, not analyzed. The asterisk denotes assessment in the yeast two-hybrid test. (All other tests were performed in transfected mammalian cells.) (F) Binding of NIK (upper panel) and of TRAF2 (lower panel) to SIVA2 involves the latter's C-terminal region (the cysteine-rich region). The indicated plasmids were co-transfected into HEK293T cells. Lysates were prepared 24 h after transfection and analyzed as indicated in the figure. To further increase SIVA2 expression in cells transfected with this plasmid alone, these cells were treated with MG132 (25 μM) during the last 4 h before harvesting. WB, anti-HIS. The deletion construct corresponding to the CRR itself was rather poorly expressed and therefore could not be used to assess the binding of proteins to this region.

FIG. 7 SIVA2 inhibits TRAF2- and NIK-mediated signaling. (A) Induction of SIVA2 in Ramos T-REx-SIVA2 cells suppresses induction of both the canonical and the alternative pathways by CD70 (middle and left panels, respectively) and of the canonical NF-κB pathway by TNF (right panel). (B) Induction of SIVA2 (left panels), but not of SIVA1 (right panels), in EcR293-CD27-SIVA2 cells suppresses activation of the alternative NF-κB pathway by CD70 (no IKBα degradation or p65 translocation to the nucleus could be discerned in CD70-treated EcR293-CD27 cells). Western blot analysis of SIVA demonstrates that the level of induction of SIVA2 is much lower than that of SIVA1. Unless further enhanced by proteasomal inhibition, SIVA2 was below detection level. (C) Suppression of SIVA increases NIK expression, and also causes constitutive activation of the alternative NF-κB pathway and increased responsiveness of the canonical pathway. Left panel: A mixture of pSUPER SIVA plasmids (2×pSUPER 275+1×pSUPER NC3) was transiently transfected into EcR293-CD27 cells expressing retrovirally transduced NIK. After 40 h the cells were treated with CD70 for 8 h, and cytoplasmic and nuclear extracts were then assayed for NF-κ proteins and NIK. Effective suppression of SIVA expression by the siRNAs was confirmed in the experiments shown in panels C, D, and E by RT-PCR of SIVA message, performed as described in Materials and Methods. Right panel: Ramos cells stably expressing lentivirally transduced SIVA shRNA NC3 (SIVA-knockdown) were treated with CD70 for the indicated time periods, and nuclear extracts were analyzed for NF-κB proteins. Oct1 served as the loading control. (D) Suppression of SIVA enhances CD70-induced NF-κB activation. HEK293T cells were transiently co-transfected with CD27, a mixture of pSUPER-SIVA plasmids, and a luciferase reporter plasmid. After 26 h the cells were treated with CD70 for 4 h. Lysates were analyzed in triplicate in two independent experiments; results represent the mean fold induction. (E) Suppression of SIVA enhances MAPK activation by CD70 and TNF. Left: Control and SIVA-knockdown Ramos cells were treated as in the right panel of C. Right: pSUPER SIVA was transiently expressed in HEK293T cells, and 48 h after transfection TNF was applied for the indicated durations. Total-cell lysates were analyzed for phosphorylated and total JNK and p38.

FIG. 8 SIVA2, cooperatively with cIAP1, mediates ubiquitination and degradation of TRAF2 in response to CD27. (A) SIVA2 facilitates ubiquitination of TRAF2 in the CD27-receptor complex. Left panel: Suppression of the recruitment of TRAF2 to the receptor complex as well as of its ubiquitination by SIVA2 knockdown. EcR293-CD27 cells were transfected with the mixture of pSUPER SIVA plasmids, and were treated 48 h later with CD70 for the indicated time periods. Western blot analysis of CD27 in the immunoprecipitated receptor complex serves as an internal control. The efficiency of SIVA suppression was evaluated in this experiment and in C by RT-PCR of SIVA message, as described in Materials and Methods. Other panels: Inducibly expressed SIVA2, but not SIVA2 (C73A) or SIVA1, enhances TRAF2 ubiquitination in the receptor complex. Ramos T-REx-SIVA2 cells, Ramos T-REx-SIVA2 (C73A) cells, or Ramos T-REx-SIVA1 cells (5×10⁷ cells) were induced with doxycycline for 2 h, and CD70 was then applied for the indicated time periods. Ubiquitin aldehyde (5 μM) was added to the cell lysates in all experiments in which protein ubiquitination in cells was assayed. (B) Suppression of SIVA blocks CD70-induced TRAF2 degradation. EcR293-CD27 cells were transfected as in the left panel of A and treated with CD70 for the indicated time periods. (C) Effect of SIVA2 on the response of NIK- or NIK (K670A)-expressing cells to CD70. EcR293-CD27-SIVA2 cells constitutively expressing retrovirally transduced NIK or NIK (K670A) mutant were treated with CD70 and, where indicated, also with ponasterone for 8 h. (D) K48-linked ubiquitination of TRAF2 in the CD27-receptor complex of cells expressing SIVA2. EcR293-CD27-SIVA2 cells were transfected with the indicated HA-tagged ubiquitin mutant plasmids and SIVA2 was induced with ponasterone for 2 h. CD70 was then applied for 15 min and the CD27-receptor complex was precipitated through anti-FLAG. The immunoprecipitate was boiled with 1% SDS, diluted 20-fold with lysis buffer, re-immunoprecipitated with anti-HA antibody, and analyzed with anti-TRAF2 antibody. (E) cIAP1 is required for CD70-induced TRAF2 degradation. EcR293-CD27 cells were transfected with cIAP1 siRNA or control siRNA and treated, 48 h after transfection, with CD70 for the indicated time periods.

FIG. 9 SIVA2 mediates ubquitination of both TRAF2 and cIAP1. (A) cIAP-1 is required for SIVA2-mediated TRAF2 ubiquitination in cells. HEK293T cells were transfected with cIAP1 siRNA and, 24 h later, with the other plasmids as indicated. The extent of ubiquitination of the TRAF2 immunoprecipitated from the lysates of these cells, as well as the cellular levels of the endogenous cIAP1 and cIAP2 and the transfected ubiquitin, were determined by western blot analyses. (B) SIVA2, but not SIVA1, enhances K48-linked polyubiquitination of TRAF2 in cells. HEK293T cells grown in 90-mm plates were transfected by the calcium phosphate method with 4 μg of FLAG-TRAF2 (C34A), together with 6 mg of HA-ubiquitin mutant plasmids and 6 μg of HIS-SIVA2 or HIS-SIVA2 (C73A) or HIS-SIVA1. The cells were lysed 24 h after transfection and TRAF2 was precipitated and analyzed as indicated. Wild-type SIVA2 and SIVA1, as well as SIVA2 (C73A) mutant, co-precipitated with TRAF2 (bottom panel). (C) SIVA2 ubiquitinates cIAP-1 in vitro. Recombinant cIAP1 was incubated with SIVA2 or the SIVA2 (C73A) mutant in a ubiquitination reaction with either UbCH5b or Ubc13/Uev1a used as the E2 enzyme. After the reaction the proteins were treated with SDS as in FIG. 8 D, then immunoprecipitated and subjected to analysis by western blotting as indicated.

### DETAILED DESCRIPTION OF THE INVENTION

The findings according to the invention show that SIVA2 is a feedback regulator of TNF/NGF receptor signaling and that modulation of SIVA2 stability can be used in therapy of disease disorder or conditions associated with the activity of these receptors.

The invention provides a stability-improved SIVA2 or salt thereof which can be used in therapy wherein said stability-improved SIVA2 is characterized by comprising one or more of the following post translation modification(s) (i) O-GlcNAcylation; (ii) phosphorylation at serine residues 5, 50, 51 of SIVA2; (iii) ubiquitination on SIVA2 residues, K17 and/or K99; or (iv) a combination of (i) to (iii). The present invention also relates to a stabilized SIVA2 mutein, isoform, fused protein, functional derivative, active fraction, fragment, circularly permutated derivative, collectively named herein stabilized SIVA2.

Among the proteins known to participate in signaling by receptors of the TNF/NGF family, it is possible to distinguish two functional groups: (i) proteins that mediate signaling, and (ii) those that regulate it, dictating which of the receptor's various activities will be turned on, at what intensity, and for how long. Proteins of the first group usually occur constitutively in the cells, ready to be recruited to the receptors upon ligand binding. Expression of those proteins that regulate signaling, however, is often itself signaling-dependent; their cellular levels are enhanced by TNF/NGF receptors, as well as by other agents that affect the function of these receptors. Earlier studies of SIVA were interpreted as suggesting that this protein mediates signaling and that it acts specifically to promote cell death. This indeed seems to be the case with SIVA1. With respect to SIVA2, it is suggested according to the invention that this protein serves rather as a regulator of signaling, not necessarily in a way that promotes cell death; and indeed, typically of proteins that regulate receptor-induced signaling, its own levels in cells are affected by signals generated by TNF/NGF receptors. Both, in its function and in the regulation of its formation, SIVA2 is shown according to the invention to differ from SIVA1. The latter, unlike SIVA2, occurs constitutively in various cells in amounts much higher than those of SIVA2, and is further induced by cellular stress. Moreover, association of SIVA1 with signaling complexes of receptors of the TNF/NGF family was not detected, nor the effects on signaling displayed by SIVA2.

SIVA2, is a short variant of SIVA1, is specifically recruited to receptors of the TNF/NGF family and can both inhibit and enhance signaling for some of their nonapoptotic effects. It was found according to the present invention that: (a) the cellular content of SIVA2, is very low in the absence of stimulation and is greatly increased after these receptors are triggered; (b) that this increase reflects its enhanced stability contributed by TRAF2 and NIK, signaling proteins that bind to SIVA2, and (c) that said enhanced stability involves post-translational modifications of SIVA2, including O-GlcNAcylation, ubiquitination in specific lysines and phosphorylation in specific serines. Also, it was found according to the invention that SIVA2 binds to and ubiquitinates the anti apoptotic protein cIAP1 and to TRAF2, triggering the latter's degradation. It was recently found by the inventors that SIVA2 also modulates ubiquitination and proteasomal processing of NIK and TRAF3 WO2007080593. In all, these findings stress that SIVA2 is a feedback regulator of TNF/NGF receptor signalling and that modulation of SIVA2 stability has a key role on signaling by receptors of the TNF/NGF family.

It was found according to the invention that the feedback loop is initiated by the recruitment of SIVA2 to the receptors' signaling complexes, as well as the dramatic stabilization of SIVA2, which can be induced by the activities of two signaling proteins TRAF2 and the protein kinase NIK, to which SIVA2 binds. Consequently, SIVA2 imposes ubiquitination of several of the signaling proteins that are recruited to the receptor and thus modulates their proteasomal processing.

Up to now, the mechanisms reported to underlie an induced increase in the cellular levels of proteins (such as A2019, TRAF120, cFLIP21, or CYLD22) that regulate signaling by receptors of the TNF/NGF family act on the transcriptional level. In contrast, it was found according to the invention that the increase in SIVA2 level following ligand stimulation occurs post-transcriptionally. Post-translational modifications of SIVA2 demonstrated according to the present invention to increase the level of SIVA2 include phosphorylation of specific serine residues, O-GlcNAcylation, and ubiquitination, and as shown according to the invention these modifications contribute to the modulation of SIVA2 stability. At least two signaling proteins seemed to participate in cytokine-induced SIVA2 stabilization: NIK, in a way that depends on its protein kinase function, and TRAF2, by a mechanism that involves its ubiquitin-ligase function.

More specifically, the following differences in SIVA1 and SIVA2 were found according to the invention; (a) SIVA2 is less expressed than SIVA1 splice variants in various cell lines and cytokines of the TNF/NGF family such as CD70, CD40L, TNF increased the amount of SIVA2 in resting PBMCs; (b) ligand activation and proteasomal inhibition, but not genotoxic stress, increase SIVA2 levels in activated PBMCs and the increase in SIVA2 levels were caused by increase in stability of SIVA2 and not by increase in SIVA2 expression; (c) The cytokine stabilization was specific for SIVA2 since that of SIVA1 remained unaltered; (d) SIVA2 is recruited to CD27 by treatment with CD70 while SIVA1 is not, in addition, SIVA2 was shown also to be recruited to CD40 and TNFR1; (e) while genotoxic agents enhance SIVA1 expression they do not affect the expression of SIVA2; (f) stabilization of SIVA2 by CD40 in cells decreased when treated with the genotoxic agent CPT. These differences of SIVA1 and SIVA2 can be advantageously used to specifically induce SIVA2 activity in therapy.

One of the modifications of SIVA2 that were found according to the invention to contribute to its stabilization in cells is phosphorylation in serine residues, particularly in every serine residues 5, 50 and 51 (3S) and especially in all serine residues 5, 21, 26, 35, 50 and 51 (6S). For example, mutations in 3S (e.g. in SIVA3SA) significantly reduced stabilization of SIVA2 and mutations 6S (SIVA6SA) almost completely reduced the stabilization of SIVA2 induced by NIK. Of note, individual serine mutations did not interfere with NIK-induced SIVA2 stabilization and tyrosine 34 of SIVA2 did not participate in its phosphorylation or stabilization by NIK. SIVA2, but only some of SIVA3SA and almost none of SIVA6SA mutants, were found to be phosphorylated also from an in-vitro kinase assay, performed with SIVA proteins immunoprecipitated from cells co-expressing NIK. Unlike NIK, it was found that TRAF2 and proteasomal inhibition do stabilize SIVA6SA mutants. Of note, serine mutations of SIVA2 compromised its stabilization by the cytokine CD40L. Also, the findings according to the invention show that lysines in SIVA2 participate in its stabilization by TRAF2. In contrast, it was found that lysines in SIVA2 are not involved in its stabilization by NIK. Another modification of SIVA2 that was found according to the invention to contribute to its stabilization in cells is O-GlcNAcylation. For example, it was found that (a) SIVA is a glycoprotein; (b) SIVA2 incorporates azido-GlcNAc in cells cotransfected with NIK and SIVA2; (c) that the β-D-N-acetyl hexosaminidase treatment abolished binding of SIVA2, extracted from cells coexpressed with NIK, to Wheat Germ Agglutinin (WGA) which selectively binds to N-Acetyl glucosamine (GlcNAc) groups and to sialic acid; (d) that inhibition of O-glycosylation interfered also with TRAF2-induced SIVA2 stabilization, but did not affect MG132-induced stabilization; (e) that inhibition of O-glycosylation blocks the NIK-mediated stabilization of wild-type SIVA2, but had no effect on the residual stabilization by NIK of the SIVA2 6SA mutant; and (f) that specific inhibition of O-GlcNAcylation blocked NIK-induced SIVA2 stabilization.

In addition, it was found according to the invention that SIVA2 ubiquitination on SIVA2 residues, K17 and/or K99 contribute to SIVA2 stabilization. This stabilization by ubiquitination appears to be induced by TRAF2.

Thus, the present invention provides the use of specific modulation of SIVA2 stability in therapy. SIVA2 modulation can be carried out or induced in vitro e.g. in cell free system, or inside the cells e.g. in vivo or ex-vivo. Modulation of SIVA2 stability can be induced in diseased cells or in cells producing unregulated levels of cytokines. Examples of cells in which modulation of SIVA2 stability can be induced include but, are not limited to, mononuclear cells, lymphoid cells, Treg cells, endothelial cells, smooth muscular cells, macrophages, lymphocytes, embryonic kidney cells, lymphoma cells, B-lymphoblastoma cell, hepatocellular liver carcinoma cell, cells expressing unregulated levels of CD27, CD40, and/or TNF receptor. In one embodiment of the invention, modulation of SIVA2 stability is induced in cells before during and/or after treatment with a genotoxic agent such as chemotherapy or irradiation.

In one embodiment of the invention, modulation of SIVA2 stability consists on increasing the stability of SIVA2. Stabilized SIVA2 is characterized by comprising one or more of the following post translation modification(s) (i) O-GlcNAcylation; (ii) phosphorylation at serine residues 5, 50, 51 of SIVA2; (iii) ubiquitination on SIVA2 residues, K17 and/or K99; or (iv) a combination of (i) to (iii).

Stabilized SIVA2 can be induced in a cell, for example, by over-expressing in the same cell one or more of the following recombinant or endogenous proteins (see EGA below) such as NIK, TRAF2, cIAP1 a ring-finger mutant of cIAP1 such as H588A, a O-GlcNAc transferase, an inhibitor of O-GlcNAcase. Stabilizaed SIVA2 can be induced in a cell by overexpressing SIVA2 together with said protein(s) e.g. as shown in the examples below. Alternatively, an activator of O-GlcNac transferase, TRAF2, inhibitor of O-GlcNAcase, inhibitor CIAP1 activity, and/or a ring-finger mutant of cIAP1 such as H588A may be used.

Stabilized SIVA2 can be used for treating, or in the manufacture of a medicament for treating a disease disorder or condition associated with low activity of SIVA2 or ameliorated by increasing the activity of SIVA2 in cells and/or in a disease; disorder; or condition in which signaling pathways activated towards protein synthesis by several members of the TNF/NGF family are associated with the pathogenesis or course of the disease disorder or condition such as e.g. cancer, an inflammatory disease, and/or an autoimmune disease. Said treating can be carried out in vivo or ex-vivo.

The term “salts” herein refers to both salts of carboxyl groups and to acid addition salts of amino groups of the polypeptide of the invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Of course, any such salts must have substantially similar activity to the SIVA2.

As used herein, the term “fragment” refers to a part or fraction of the polypeptide molecule, provided that the shorter peptide retains the desired biological activity of SIVA2. Fragments may readily be prepared by removing amino acids from either end of the polypeptide and testing the biological activity of the resulting fragment for example: binding to cIAP1, binding to TRAF2, induction of NIK degradation, and/or inhibition of NIK-mediated NFκB activation in cells. Proteases that remove one amino acid at a time from either the N-terminal or the C-terminal of a polypeptide are known in the art, and fragments that retain the desired biological activity can be obtaining as a matter of routine experimentation by employing such proteases.

As “active fractions” of the protein the present invention refers to any fragment or precursor of the polypeptidic chain of the compound itself, alone or in combination with related molecules or residues bound to it, for example residues of sugars or phosphates, or aggregates of the polypeptide molecule when such fragments or precursors show the same activity of SIVA2 as medicament. “Precursors” are compounds which can be converted into the SIVA2 in the human or animal body.

The definition “functional derivatives” as herein used refers to derivatives which can be prepared from the functional groups present on the lateral chains of the amino acid moieties or on the terminal N- or C-groups according to known methods and are comprised in the invention when they are pharmaceutically acceptable i.e. when they do not destroy the protein activity or do not impart toxicity to the pharmaceutical compositions containing them. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups. SIVA2 may be conjugated to polymers in order to improve the properties of the protein, such as the stability, half-life, bioavailability, tolerance by the human body, or immunogenicity. Therefore, one embodiment of the invention relates to a functional derivative of SIVA2 comprising at least one moiety attached to one or more functional groups, which occur as one or more side chains on the amino acid residues. One embodiment of the invention relates to SIVA2 polypeptide linked to Polyethlyenglycol (PEG). PEGylation may be carried out by known methods, such as the ones described in WO 92/13095, for example.

The term “circularly permuted derivatives” as used herein refers to a linear molecule in which the termini have been joined together, either directly or through a linker, to produce a circular molecule, and then the circular molecule is opened at another location to produce a new linear molecule with termini different from the termini in the original molecule. Circular permutations include those molecules whose structure is equivalent to a molecule that has been circularized and then opened. Thus, a circularly permuted molecule may be synthesized de novo as a linear molecule and never go through a circularization and opening step. The preparation of circularly permutated derivatives is described in WO95/27732.

As used herein the term “muteins” refers to analogs of SIVA2. The present invention also concerns analogs of the above SIVA2 protein of the invention, which analogs retain essentially the same biological activity of the SIVA2 protein having essentially only the naturally occurring sequences of SIVA2. Such “analogs” may be ones in which up to about 30 amino acid residues may be deleted, added or substituted by others in the SIVA2 protein, such that modifications of this kind do not substantially change the biological activity of the protein analog with respect to the protein itself. Thus, one or more of the amino acid residues of the naturally occurring components of SIVA2 are replaced by different amino acid residues, or are deleted, or one or more amino acid residues are added to the original sequence of SIVA2, without changing considerably the activity of the resulting products as compared with the original SIVA2. These muteins are prepared by known synthesis and/or by site-directed mutagenesis techniques, or any other known technique suitable therefore.

Any such mutein preferably has a sequence of amino acids sufficiently duplicative of that of the basic SIVA2 such as to have substantially similar activity thereto. Thus, it can be determined whether any given mutein has substantially the same activity as the basic SIVA2 of the invention by means of routine experimentation comprising subjecting such an analog to the biological activity tests set forth in Examples below e.g. monitoring binding to cIAP1, binding to TRAF2, binding to NIK, induction of NIK degradation, ubiquitination of TRAF2, self ubiquitination, ubiquitination of cIAP1 or inhibition of NIK-mediated NFxB activation in cells.

Muteins of the SIVA2 protein which can be used in accordance with the present invention, or nucleic acid coding therefore, include a finite set of substantially corresponding sequences as substitution peptides or polynucleotides which can be routinely obtained by one of ordinary skill in the art, without undue experimentation, based on the teachings and guidance presented herein. For a detailed description of protein chemistry and structure, see Schulz, G. E. et al., Principles of Protein Structure, Springer-Verlag, New York, 1978; and Creighton, T. E., Proteins: Structure and Molecular Properties, W.H. Freeman & Co., San Francisco, 1983, which are hereby incorporated by reference. For a presentation of nucleotide sequence substitutions, such as codon preferences, see. See Ausubel et al., Current Protocols in Molecular Biology, Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989.

Preferred changes for muteins in accordance with the present invention are what are known as “conservative” substitutions. Conservative amino acid substitutions of those in the SIVA2 protein having essentially the naturally-occurring SIVA2 sequences, may include synonymous amino acids within a group which have sufficiently similar physicochemical properties that substitution between members of the group will preserve the biological function of the molecule, Grantham, Science, Vol. 185, pp. 862-864 (1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues, Anfinsen, “Principles That Govern The Folding of Protein Chains”, Science, Vol. 181, pp. 223-230 (1973). Analogs produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser. Ala, Gly, His, Gln, Thr |
| Ala | Gly, Thr, Pro, Ala |
| Val | Met, Tyr, Phe, Ile, Leu, Val |
| Gly | Ala, Thr, Pro, Ser. Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonimous Amino Acids

| Amino Acid | Synonymous Group |
|---|---|
| Ser | Ser |
| Arg | His, Lys, Arg |
| Leu | Ile, Phe, Met, Leu |
| Pro | Ala, Pro |
| Thr | Thr |
| Ala | Pro, Ala |
| Val | Met, Ile, Val |
| Gly | Gly |
| Ile | Ile, Met, Phe, Val, Leu |
| Phe | Met, Tyr, Ile, Leu, Phe |
| Tyr | Phe, Tyr |
| Cys | Ser, Cys |
| His | Arg, Gln, His |
| Gln | Glu, His, Gln |
| Asn | Asp, Asn |
| Lys | Arg, Lys |
| Asp | Asn, Asp |
| Glu | Gln, Glu |
| Met | Phe, Ile, Val, Leu, Met |
| Trp | Trp |

TABLE III
Most Preferred Groups of Synonymous Amino Acids

| Amino Acid | Synonymous Group |
|---|---|
| Ser | Ser |
| Arg | Arg |
| Leu | Ile, Met, Leu |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Ser, Cys |
| His | His |
| Gln | Gln |
| Asn | Asn |
| Lys | Lys |
| Asp | Asp |
| Glu | Glu |
| Met | Ile, Leu, Met |
| Trp | Trp |

Examples of production of amino acid substitutions in proteins which can be used for obtaining muteins of SIVA2 include any known method steps, such as presented in U.S. Pat. Nos. RE 33,653, 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Straw et al).

In another preferred embodiment of the present invention, any mutein of the SIVA2 protein for use in the present invention has an amino acid sequence essentially corresponding to that of the above noted SIVA2 protein of the invention. The term “essentially corresponding to” is intended to comprehend muteins with minor changes to the sequence of the basic SIVA2 protein which does not affect the basic characteristics thereof, particularly insofar as its ability to SIVA2 is concerned. The type of changes which are generally considered to fall within the “essentially corresponding to” language are those which would result from conventional mutagenesis techniques of the DNA encoding the SIVA2 protein of the invention, resulting in a few minor modifications, and screening for the desired activity in the manner discussed above.

In one embodiment of the invention, any such mutein has at least 40% identity with the sequence of SIVA2, more preferably, it has at least 50%, at least 60%, at least 70%, at least 80% or, most preferably, at least 90% identity thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

The term “sequence identity” as used herein means that the amino acid sequences are compared by alignment according to Hanks and Quinn (1991) with a refinement of low homology regions using the Clustal-X program, which is the Windows interface for the ClustalW multiple sequence alignment program (Thompson et al., 1994). The Clustal-X program is available over the internet at ftp://ftp-igbmc.u-strasbg.fr/pub/clustalx/. Of course, it should be understood that if this link becomes inactive, those of ordinary skill in the art can find versions of this program at other links using standard internet search techniques without undue experimentation. Unless otherwise specified, the most recent version of any program referred herein, as of the effective filing date of the present application, is the one which is used in order to practice the present invention.

If the above method for determining “sequence identity” is considered to be nonenabled for any reason, then one may determine sequence identity by the following technique. The sequences are aligned using Version 9 of the Genetic Computing Group's GDAP (global alignment program), using the default

(BLOSUM62) matrix (values −4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence.

Muteins in accordance with the present invention include those encoded by a nucleic acid, such as DNA or RNA, which hybridizes to DNA or RNA under stringent conditions and which encodes a SIVA2 protein in accordance with the present invention, comprising essentially all of the naturally-occurring sequences encoding SIVA2. For example, such a hybridizing DNA or RNA may be one encoding the same protein of the invention having, for example, the sequence of SIVA2, but which nucleotide differs in its nucleotide sequence from the naturally-derived nucleotide sequence by virtue of the degeneracy of the genetic code, i.e., a somewhat different nucleic acid sequence may still code for the same amino acid sequence, due to this degeneracy.

The findings according to the invention allow the preparation of stabilized SIVA2. For example, stabilized SIVA2 or stability improved SIVA2 may be obtained by increasing O-GlcNAcylation in the protein, for example by contacting the protein with O-GlcNAc transferase and/or by inhibiting the activity of O-GlcNAcase. Induction of GlcNAc transferase can be aimed, for example, by increasing the levels of UDP-GlcNAc in cells (Slawson et al., Journal of cellular Biochemistry 97:71-83, 2006). Also, stabilization of SIVA2 may be obtained or further increased by inducing phosphorylation at serine residues 5, 50, and 51 of the protein. Increased stabilization may be obtained by phosphorylation of serine residues 5, 50, 51, 21, 26, and 35, for example by contacting SIVA2 with NIK. In addition, stabilization of SIVA2 may be obtained or further increased by increasing ubiquitination of SIVA2, for example by contacting the protein with TRAF2. Lysine residues involved in SIVA2 stabilization by ubiquitination are either one of two of the residues, K17 and K99. Mutations in these residues did not affect the stabilization of SIVA2 by NIK. Another way to stabilize SIVA2 is by contacting SIVA2 with a ring-finger mutant of cIAP1 such as H588A. Said contacting of SIVA2 with the other mentioned proteins can be carried out in vivo (e.g. inside cells) and in vitro (e.g. in a cell free system). In one embodiment of the invention, for specifically increasing stability of SIVA2, cells can be manipulated to overexpress one or more of the following proteins O-GlcNAc transferase, NIK, TRAF2, or a ring-finger mutant of cIAP1 such as H588A. Overexpression of endogenous protein can be carried out, for example, by endogenous gene activation (EGA, see bellow). Overexpression of exogenous protein can be carried out, by introducing the gene encoding the protein into the cells, for example, by using an expression vector (see below). In a further embodiment of the invention SIVA2 is co-overexpressed with the protein(s). If the stabilized SIVA2 is prepared ex-vivo the cells are cultured under conditions allowing production of said stability-improved SIVA2 and recovering the resulting SIVA2 from the culture. For example, cells stressed with a nutrient poor or nutrient-exessive environment were shown to elevate O-GlcNac levels and can be used to produce stabilized SIVA2 Also, all forms of stress tested (osmotic, ethanolic, oxidative, and heat schok) to date rapidly raise O-GlcNac levels in cells (Slawson et al., 2006).

Furthermore, the invention provides a host cell comprising stabilized SIVA2 selected from eukaryotic cells, such as a mammalian, insect, and yeast cells. In one embodiment of the invention the cells are HeLa, 293 THEK or CHO cells. Alternatively, the invention provides a method of producing s stabilized SIVA2 of the invention comprising the generation of a transgenic animal and isolating the protein produced from the body fluids of the animal.

Stabilized SIVA2 can be produced in eukaryotic host cells transfected, transformed or infected with vectors encoding SIVA2, or in transgenic animals. When using transgenic animals, it is particularly advantageous to produce heterologous polypeptides in their milk.

Overexpression of a protein in a mammalian cell may be carried out by inserting the DNA encoding the polypeptide into a vector comprising a promoter, optionally an intron sequence and splicing donor/acceptor signals, and further optionally comprising a termination sequence and signal peptide for secretion, by well-known techniques (for example, as described in Current Protocols in Molecular Biology, chapter 16).

Overexpression of a protein in a mammalian cell may be carried out by inducing increase in expression of the endogenous gene which encodes e.g. SIVA2 polypeptide and/or O-GlcNAc transferase, NIK, and TRAF2. Altering expression of endogenous SIVA2 and/or O-GlcNAc transferase and/or O-GlcNAc transferase, NIK, and TRAF2 can be also employed. If desired, a compound may increase the level of expression of the gene or the activity of endogenous protein. Such compound can be a vector for inducing the endogenous production of a protein in a cell which expresses amounts of the protein which are not sufficient. The vector may comprise regulatory sequences functional in the cells desired to express the protein. Such regulatory sequences may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “Endogenous Gene Activation” (EGA), and it is described e.g. in WO 91/09955.

It will be understood by the person skilled in the art that it is also possible to shut peptide expression directly, in situations in which a peptide is over-expressed and results in excessive amounts of the polypeptide in a cell or when it is desired to shut the peptide expression. For example, to increase O-GlcNAcidated SIVA2 in a cell the expression of O-GlcNAcase may be ablated by EGA. To do that, a negative regulation element, like e.g. a silencing element, may be introduced into the gene locus of the protein, thus leading to down-regulation or prevention of protein expression. The person skilled in the art will understand that such down-regulation or silencing of protein expression has the same effect as the use of an inhibitor.

The invention also provides a pharmaceutical composition comprising a pharmaceutically acceptable carrier and a stability-improved SIVA2 or salt thereof characterized by comprising one or more of the following post translation modification(s) (i) O-GlcNAcylation; (ii) phosphorylation at serine residues 5, 50, 51 of SIVA2; (iii) ubiquitination on SIVA2 residues, K17 and/or K99; or (iv) a combination of (i) to (iii).

It was found according to the invention that SIVA2 binds to various other proteins known to mediate signaling by receptors of the TNF/NGF family such as TRAF2, cIAP1 and NIK. TRAF2, like NIK binds to CRR in SIVA2 and cIAP1 was found according to the invention to bind to the N-terminal part of SIVA2 (upstream of the CRR). It was also found that SIVA2 can inhibit TRAF2 and NIK mediated signaling since induction of SIVA2 suppresses the activation of both the alternative and the canonical NF-κB pathways by CD70 as well as activation of the canonical pathway by TNF. SIVA1, on the other hand, although expressed at much higher level than SIVA2, had no such effect. Conversely, cells in which SIVA expression has been knocked down displayed constitutive activation of the alternative NF-κ pathway and also displayed somewhat increased basal levels of canonical NF-κB pathway and heightened responsiveness of this pathway to activation by CD70. Knockdown of SIVA also enhanced the induction of JNK and p38 kinase phosphorylation both by CD70 and by TNF. SIVA2, cooperatively with cIAP1, mediated ubiquitination and degradation of TRAF2 in response to CD27. Also, it was previously reported that TRAF2 molecules recruited to CD27 are massively ubiquitinated (Ramakrishnan et al., 2004). Knockdown of SIVA attenuated the CD70 ubiquitination of TRAF2. In contrast, induction of SIVA2 but not SIVA1, enhanced it.

It was previously found that SIVA2 possesses intrinsic ubiquitin-ligase activity and that, SIVA2 facilitated in-vitro ubiquitination of TRAF2 (WO2007080593). It was found that cysteine residue at position 73 within the CRR in SIVA2 was needed for the ubiquitination of TRAF2. It was demonstrated using transfected cells, that over-expression of wild-type SIVA2, but not SIVA1, markedly increased the K48-linked (though not the K63-linked) polyubiquitination of TRAF2 beyond that observed when TRAF2 was expressed alone, whereas SIVA2 (C73A) hardly affected the ubiquitination. Self-ubiquitination of SIVA2 in vitro was not affected by this mutation, but it was drastically reduced by complete deletion of the CRR. Expression of SIVA2 C73A mutant in cells was found to ablate the enhancing effect of SIVA2 on the ubiquitination of TRAF2 in the CD27 complex.

In addition to TRAF2, it was found according to the invention that cIAP1 was also effectively ubiquitinated by SIVA2, and that this ubiquitination too was compromised by the SIVA2 (C73A) mutation. Knockdown of cIAP1, dramatically reduced the ubiquitination of TRAF2 in response to SIVA2 expression. Thus, although SIVA2 has the ability to directly ubiquitinate TRAF2 in vitro, its facilitation of TRAF2 ubiquitination within cells is either mediated through enhancement of the ability of cIAP1 to do so, or requires cIAP1 to play a permissive role. Triggering of CD27 resulted in a significant decrease in the cellular amounts of TRAF2, suggesting that its ubiquitination within the receptor complex targets for degradation. The ubiquitin chains whose ligation to TRAF2 was facilitated by SIVA2 were primarily K48-linked, as is generally the case with ubiquitination that prompts proteosomal degradation, rising the possibility that this SIVA2 effect contributes to the induction of TRAF2 degradation by CD27. Consistently, knockdown of SIVA expression prevented the downregulation of TRAF2 by CD27, whereas induction of SIVA2 enhanced it.

As mentioned above, knockdown of SIVA also resulted in constitutive activation of the alternative NF-κB pathway. Suppression of cIAP1 expression also results in NF-κB activation (Varfolomeev et al., 2007) (Vince et al., 2007); in addition, it compromises the downregulation of TRAF2 by TNF-RII, another receptor of the TNF/NGF family (Li et al., 2002). According to the present invention knockdown of cIAP1 (like knockdown of SIVA2) compromised the downregulation of TRAF2 by CD27 as well, along with constitutive activation of the alternative NF-κB pathway. These findings suggested that SIVA2 and cIAP1 play a shared role in the induction of TRAF2 degradation by CD27 and in the regulation of NF-κB.

In view of these findings regarding the function of SIVA2, the specific modulation of SIVA2 stabilization can be used in therapy (prevention or treatment) or diagnosis of situations associated with the level or activity of SIVA2 and/or in situations in which signaling pathways activated towards protein synthesis by several receptors members of the TNF/NGF family, and particularly those that activate the alternative pathway, and are associated with the pathogenesis or course of the situation.

Thus, in one embodiment of the invention, stabilized SIVA2 is useful in modulating the activity of NIK and NF-κB for example, wherein the disease, disorder, or condition is characterized by inappropriate NIK-mediated activity or NIK-mediated NF-κB activity such as for example in developmental disorders, cell proliferative disorders and immune disorders. In one embodiment, the disease, disorder, or condition is characterized by increased host immune, inflammatory response and/or cell proliferation mediated by increased NIK and NF-κB activity and thus a stabilized SIVA2 may be used to treat said disease.

Such situations in which modulation of SIVA2 stability is beneficial may include diseases disorders or conditions such as developmental disorders; cell proliferative disorders for example neoplastic disorders, like cancer, melanoma, sarcoma, renal tumour, colon tumour; genetic disorders; nervous system disorders; metabolic disorders; infections and other pathological conditions; immune disorders such as osteoarthritis, autoimmune disease, rheumatoid arthritis, psoriasis, systemic multiple sclerosis, and lupus erythematosus; inflammatory disorders such as glomerulonephritis, allergy, rhinitis, conjunctivitis, uveitis, digestive system inflammation, inflammatory bowel disease such as Crohn's disease and ulcerative colitis, myasthenia gravis, pancreatitis, sepsis, endotoxic shock, cachexia, myalgia, ankylosing spondylitis, asthma, airway inflammation; wound healing; dermatological disease; ageing; and infections, including plasmodium, bacterial infection and viral infection. Thus, stabilized SIVA2 may be used in the manufacture of a medicament for the treatment of such situations.

In one embodiment of the invention, diseases, disorders or conditions associated with decreased SIVA2, increased NIK activity and/or NF-κB activity in cells such as such as malignancies, including both primary tumor and metastasis, asthma, rheumatoid arthritis, atherosclerosis, inflammation may be treated by administering stabilized SIVA2 of the invention capable of downregulating/inhibiting the activity of NIK and/or NF-κB activity in cells.

The invention allows modulation of SIVA2 stability to modulate its activity in cells and in order to modulate/mediate intracellular effects on the inflammation, cell death or cell survival pathways in which activity of SIVA2 is involved directly, or indirectly via other modulators/mediators of TNF/NGF pathways. To modulate SIVA2 activity, cells can be treated by introducing into said cells said stabilized SIVA2 or by inducing modulation of SIVA2 within the cells. In one embodiment of the invention, a SIVA2 polynucleotide is carried in a suitable vector which is capable of effecting the insertion of said polynucleotide into said host cells in a way that said sequence is expressed in said cells. The vector can be a virus vector carrying also a sequence encoding an enzyme capable of increasing GlcNAc moiety in proteins such as O-GlcNAc transferase, and/or NIK, TRAF2, a ring-finger mutant of cIAP1 (H588A), or an inhibitor of GlcNAcidase to stabilize the expressed SIVA2. The treatment can be effected by infecting said cells with said vector. Of advantage is overexpressing SIVA2 in vivo in specific pathologies or under specific conditions in which O-GlcNac transferase (OGT) expression or activity is high or O-GlcNacase expression or activity is reduced resulting in proteins exhibiting increased O-GlcNAc content. An example of such pathologies in which cells have increased O-GlcNac content is in Type II diabetes (Slawson et al., 2006). Of advantage is overexpressing SIVA2 in vivo together with OGT induction e.g. by stressing the cells to be treated e.g. by hypothermic conditions (Slawson et al., 2006).

Reducing stabilization of SIVA2 can be achieved, to treat disease disorder or conditions, associated with increased levels of SIVA, decreased activity of NF-κB or NIK, when increase of Levels of NF-κB or NIK in cells is desired, wherein said reducing in SIVA2 stabilization is carried out by decreasing the following post translation modification(s) of SIVA2 (i) O-GlcNAcylation; (ii) phosphorylation at serine residues 5, 50, 51 of SIVA2; (iii) ubiquitination; or (iv) a combination of (i) to (iii). Reducing stability of SIVA2 can be achieved by decreasing phosphorylation in serine residues 5, 50 and 51 (3S) and especially in serine residues 5, 21, 26, 35, 50 and 51 (6S) of SIVA2, e.g. by mutating this residues such as in SIVA3SA and SIVA6SA mutants; or by using these mutants to compete with SIVA2 activity, inhibition of O-glycosylation and specific inhibition of O-GlcNAcylation e.g. by reducing O-GlcNAcylation e.g. by β-D-N-acetyl hexosaminidase treatment, decreasing O-GlcNAc transferase activity, activation of O-GlcNAcase, reduction of the level of UDP-GlcNAc, and using cIAP1. Reducing the stability of SIVA2 may be used e.g. for busting the immune response such as in immunocompromosed subjects. Also, reducing the stability of SIVA2 may be used in ischemia/reperfision, since this condition is accompanied by increase in levels of SIVA (Padanilam et al., 1998). Decrease of SIVA2 stability may be used to when decrease apoptosis is desired, for example to decrease apoptosis in normal cells.

The invention provides a method and/or kit for diagnosing a disease in a subject comprising assessing SIVA2 post translation modification(s) (i) O-GlcNAcylation; (ii) phosphorylation at serine residues 5, 50, 51 of SIVA2; (iii) ubiquitination on SIVA2 residues, K17 and/or K99; or (iv) a combination of (i) in tissue from said subject and comparing said level of post translation modification to a control level. The control level can be the level in a healthy individual. A level of SIVA2 post translation modification(s) in a subject that is different to that of said control level is indicative of disease. Also, the invention provides a similar method for monitoring the therapeutic treatment of disease in a patient by monitoring the level of post translation modification(s) in tissue from a patient before, after and/or during the therapeutic treatment. A level of SIVA2 post translation modification(s) in a patient after therapeutic treatment that is different to that of the patient before therapeutic treatment is indicative of usefulness of the therapy. The SIVA2 post translation modification(s) in the tissue can be measured, for example as shown in the examples below. Using specific methods and kits of the invention, SIVA2 post translation modification(s) may be used to find association of the levels of SIVA2 post translation modification(s) with a human disease, disorder or condition that may then be prevented, treated or alleviated by administrating an agent that is capable of regulating SIVA2 post translation modification(s).

A therapeutic or diagnostic or research-associated use of some of these tools necessitates their introduction into cells of a living organism. For this purpose, it is desired to improve membrane permeability of peptides, proteins and oligonucleotides. Derivatization with lipophilic structures, may be used in creating peptides and proteins with enhanced membrane permeability. For instance, the sequence of a known membranotropic peptide as noted above may be added to the sequence of the peptide or protein. Further, the peptide or protein may be derivatized by partly lipophilic structures such as the above-noted hydrocarbon chains, which are substituted with at least one polar or charged group. For example, lauroyl derivatives of peptides have been described by Muranishi et al., 1991 (Lipophilic peptides: synthesis of lauroyl thyrotropin-releasing hormone and its biological activity. Pharm Res. 1991 May; 8(5):649-52). Further modifications of peptides and proteins comprise the oxidation of methionine residues to thereby create sulfoxide groups, as described by Zacharia et al. 1991 (Eur J Pharmacol. 1991 Oct. 22; 203(3):353-7). Zacharia and co-workers also describe peptide or derivatives wherein the relatively hydrophobic peptide bond is replaced by its ketomethylene isoester (COCH2). These and other modifications known to the person of skill in the art of protein and peptide chemistry enhance membrane permeability.

Another way of enhancing membrane permeability is the use receptors, such as virus receptors, on cell surfaces in order to induce cellular uptake of the peptide or protein. This mechanism is used frequently by viruses, which bind specifically to certain cell surface molecules. Upon binding, the cell takes the virus up into its interior. The cell surface molecule is called a virus receptor. For instance, the integrin molecules CAR and AdV have been described as virus receptors for Adenovirus, see Hemmi et al. 1998 (Hum Gene Ther. 1998 Nov. 1; 9(16):2363-73), and references therein. The CD4, GPR1, GPR15, and STRL33 molecules have been identified as receptors/co-receptors for HIV, see Edinger et al. 1998 (Virology. 1998 Sep. 30; 249(2):367-78) and references therein.

Thus, conjugating peptides, proteins or oligonucleotides to molecules that are known to bind to cell surface receptors will enhance membrane permeability of said peptides, proteins or oligonucleotides. Examples for suitable groups for forming conjugates are sugars, vitamins, hormones, cytokines, transferrin, asialoglycoprotein, and the like molecules. Low et al., U.S. Pat. No. 5,108,921, describes the use of these molecules for the purpose of enhancing membrane permeability of peptides, proteins and oligonucleotides, and the preparation of said conjugates.

Low and co-workers further teach that molecules such as folate or biotin may be used to target the conjugate to a multitude of cells in an organism, because of the abundant and unspecific expression of the receptors for these molecules.

The above use of cell surface proteins for enhancing membrane permeability of a peptide, protein or oligonucleotide of the invention may also be used in targeting said peptide, protein or oligonucleotide of the invention to certain cell types or tissues. For instance, if it is desired to target cancer cells, it is preferable to use a cell surface protein that is expressed more abundantly on the surface of those cells. Examples are the folate receptor, the mucin antigens MUC1, MUC2, MUC3, MUC4, MUC5AC, MUC5B, and MUC7, the glycoprotein antigens KSA, carcinoembryonic antigen, prostate-specific membrane antigen (PSMA), HER-2/neu, and human chorionic gonadotropin-beta. The above-noted Wang et al., 1998 (J Control Release. 1998 Apr. 30; 53(1-3):39-48. Review), teaches the use of folate to target cancer cells, and Zhang et al. 1998 (Clin Cancer Res. 1998 November; 4(11):2669-76. and Clin Cancer Res. 1998 February; 4(2):295-302), teaches the relative abundance of each of the other antigens noted above in various types of cancer and in normal cells.

SIVA2 and/or proteins capable of modifying its stability may therefore, using the above-described conjugation techniques, be targeted to certain cell type as desired. For instance, if it is desired to inhibit NIK in cells of the lymphocytic lineage, polypeptide or polynucleotide or compounds of the invention SIVA2 may be targeted at such cells, for instance, by using the MHC class II molecules that are expressed on these cells. This may be achieved by coupling an antibody, or the antigen-binding site thereof, directed against the constant region of said MHC class II molecule to the protein or peptide of the invention. Further, numerous cell surface receptors for various cytokines and other cell communication molecules have been described, and many of these molecules are expressed with in more or less tissue- or cell-type restricted fashion. Thus, when it is desired to target a subgroup of T cells, the CD4 T cell surface molecule may be used for producing the conjugate of the invention. CD4-binding molecules are provided by the HIV virus, whose surface antigen gp42 is capable of specifically binding to the CD4 molecule.

In one embodiment, peptides and polynucleotides may be introduced into cells by the use of a viral vector. The use of vaccinia vector for this purpose is detailed in chapter 16 of Current Protocols in Molecular Biology. The use of adenovirus vectors has been described e.g. by Teoh et al. (Blood. 1998 Dec. 15; 92(12):4591-601), Narumi et al, 1998 (Blood. 1998 Aug. 1; 92(3):822-33; and Am J Respir Cell Mol Biol. 1998 December; 19(6):936-41), Pederson et al, 1998 (J Gastrointest Surg. 1998 May-June; 2(3):283-91), Guang-Lin et al., 1998 (Transplant Proc. 1998 November; 30(7):2923-4), and references therein, Nishida et al., 1998 (Spine. 1998 Nov. 15; 23(22):2437-42; discussion 2443), Schwarzenberger et al 1998 (J Immunol. 1998 Dec. 1; 161(11):6383-9), and Cao et al., 1998 (Gene Ther. 1998 August; 5(8):1130-6). Retroviral transfer of antisense sequences has been described by Daniel et al. 1998 (J Biomed Sci. 1998 September-October; 5(5):383-94).

When using viruses as vectors, the viral surface proteins are generally used to target the virus. As many viruses, such as the above adenovirus, are rather unspecific in their cellular tropism, it may be desirable to impart further specificity by using a cell-type or tissue-specific promoter. Griscelli et al., 1998 (Hum Gene Ther. 1998 Sep. 1; 9(13):1919-28) teach the use of the ventricle-specific cardiac myosin light chain 2 promoter for heart-specific targeting of a gene whose transfer is mediated by adenovirus.

Alternatively, the viral vector may be engineered to express an additional protein on its surface, or the surface protein of the viral vector may be changed to incorporate a desired peptide sequence. The viral vector may thus be engineered to express one or more additional epitopes, which may be used to target, said viral vector. For instance, cytokine epitopes, MHC class II-binding peptides, or epitopes derived from homing molecules may be used to target the viral vector in accordance with the teaching of the invention. SIVA2 and proteins capable of modulating its stability can be targeted by introducing a promoter capable of selective expression in specific cells.

The findings according to the invention show that when receptors of the TNF/NGF family are triggered, SIVA2 is stabilized (by TRAF2 and NIK), and this results in an increase in SIVA2 cellular level. When this increase in SIVA2 levels occurs, SIVA2 binds to TRAF2, cIAP1 and NIK. As a result of this binding and SIVA2 E3 activity, TRAF2 is downregulated. As a result of downregulation of TRAF2, signaling by the receptors (signaling for activation of both the canonical and alternative pathway, as well as signaling for JNK and p38 MAP kinases) is arrested. Therefore, both molecules which block the stabilization of SIVA2, and molecules capable of blocking the interaction of SIVA2 with cIAP1 or TRAF2 will induce prolongation of signaling by receptors of the TNF/NGF family. A possible use of such prolongation of signaling by receptors of the TNF/NGF family is for potentiation of immune functions such as raising antibodies. Examples of subjects in which it may be desired to obtain such prolongation of signaling by receptors of the TNF/NGF family are AIDS patients, immunopsuppressed cancer patients, and in elderly people. Conversely, molecules capable of facilitating the stabilization of SIVA2 or its interaction with cIAP1 or TRAF2 will downregulate signaling by receptors of the TNF/NGF family. Examples in which it will be desired to induce downregulation of signaling by receptors of the TNF/NGF family are automimmune diseases such as SLE RA etc., or kidney ischemia. Thus, the invention provides complexes of SIVA2 with TRAF2 and SIVA2 with cIAP1 and use of these complexes for screening molecules capable of modulating signaling by receptors of the TNF/NGF family in a disease, disorder or condition.

In one embodiment, the invention provides a method for screening a molecule capable modulating signaling by members of the TNF/NGF receptor family in a disease, disorder or condition comprising contacting SIVA2 with cIAP or TRAF2, monitoring the level of the complex of SIVA2 with cIAP or TRAF2 in the presence and in the absence of a candidate molecule, wherein a change in the level of SIVA2-cIAP or SIVA2-TRAF2 complex in the presence of a candidate molecule is indicative that the candidate molecule modulates signaling by the members of the TNF/NGF receptor family.

In another aspect, the invention provides a method for screening a molecule capable modulating signaling by members of the TNF/NGF receptor family in a disease, disorder or condition comprising inducing SIVA2 stability in the presence and in the absence of a candidate molecule, wherein a change in the level of stabilized SIVA2 in the presence of a candidate molecule is indicative that the candidate molecule modulates signaling by the members of the of the TNF/NGF receptor family.

Molecules screened in such method(s) and found to block the stabilization of SIVA2, and found to be capable of blocking the interaction of SIVA2 with cIAP1 or TRAF2 will be useful in prolongation of signaling by members of the TNF/NGF receptor family. Conversely, molecules screened in such assays and found to be capable of facilitating the stabilization of SIVA2 or its interaction with cIAP1 or TRAF2 will be useful to downregulate signaling by members of the TNF/NGF receptor family.

Examples of assays monitoring the levels of SIVA2 with cIAP1 or TRAF2 and assays monitoring SIVA2 stability are provided in the Examples below.

Examples of candidate molecules that can be screened in the screening methods of the invention include, but are not limited to, small organic molecules, peptides (e.g. antibodies), nucleic acids, and molecules from natural extracts, carbohydrates or any other substance. Test agents include synthetic organic compounds created e.g. by combinatorial chemistry. The compounds tested may be obtained not only through combinatorial chemistry, but also by other high throughput synthesis methods. Automated techniques enable the rapid synthesis of libraries of molecules, large collections of discrete compounds, which can be screened. Producing larger and more diverse compound libraries increases the likelihood of discovering a useful drug within the library. For high throughput screening robots can be used to test thousands of molecules.

The compositions according to the invention can be administered to a patient in a variety of ways. Any suitable route of administration is envisaged by the invention such as, but not limited to, intraliver, intradermal, transdermal (e.g. in slow release formulations), intramuscular, intraperitoneal, intravenous, subcutaneous, oral, epidural, topical, and intranasal routes. The composition can be administered together with other biologically active agents.

The definition of “pharmaceutically acceptable” is meant to encompass any carrier, which does not interfere with effectiveness of the biological activity of the active ingredient and that is not toxic to the host to which it is administered. For example, for parenteral administration, the substance according to the invention may be formulated in a unit dosage form for injection in vehicles such as saline, dextrose solution, serum albumin and Ringer's solution.

A “therapeutically effective amount” is such that when administered, the said substances of the invention induce a beneficial effect in therapy. The dosage administered, as single or multiple doses, to an individual may vary depending upon a variety of factors, including the route of administration, patient conditions and characteristics (sex, age, body weight, health, and size), extent and severity of symptoms, concurrent treatments, frequency of treatment and the effect desired. Adjustment and manipulation of established dosage ranges are well within the ability of those skilled in the art.

The term “dosage” relates to the determination and regulation of the frequency and number of doses.

All references cited herein, including articles or abstracts, published or unpublished patent application, issued patents or any other references, are entirely incorporated by reference herein.

The invention will be now illustrated by the following non-limiting examples.

### EXAMPLES

### Material and Methods

Reagents. mCD70, hCD40L, were produced by large-scale transfection of human embryonic kidney HEK-293T cells with the relevant expression constructs (see below). Tumor necrosis factor (TNF), a gift from Dr. G. Adolf, Boehringer Institute, Vienna, Austria, was applied to cells at a concentration of 100 ng/ml. Phytohemagglutinin (PHA), 6-diazo-5-oxo-L-norleucine (DON), camptothecin (CPT), N-acetyl-D-glucosamine and cisplatin (CIS) were purchased from Sigma. MG132, benzyl-α-GalNAc (BADGP), lactacystin and ponasterone were from purchased from Calbiochem. Puromycin was from Invitrogen, Agarose-bound wheat-germ agglutinin (WGA) was purchased from Vector Laboratories, and β-D-N-acetyl hexosaminidase was purchased from V-Labs. E1 and E2 enzymes were from Boston Biochem and from Alexis Biochemicals. [32P]orthophosphate was from Amersham Biosciences, streptavidin HRP was from Pierce.

Cells. Peripheral-blood mononuclear cells (PBMCs) were isolated from buffy-coat samples and cultured as described (Ramakrishnan et al., 2004). Ecdysone-inducible EcR-293-CD27 cell lines expressing SIVA2 (EcR-293-CD27-SIVA2) or SIVA1 (EcR-293-CD27-SIVA1) and EcR-293-CD40 were generated by transfection using the calcium phosphate method according to the instructions of the manufacturer (Invitrogen). All adherent cells—HEK-293T, EcR-293 (Invitrogen), HeLa, HeLa T-REx (Invitrogen), and HepG2—were cultured in Dulbecco's modified Eagle's medium. Both culture media were supplemented with 10% fetal calf serum, 100 U/ml pencillin, and 100 μg/ml streptomycin. The human lymphoblastoid lines Ramos (Human Burkitt's lymphoma cell line) and BJAB (B-lymphoblastoma cell line) were cultured in RPMI medium. Ecdysone-inducible EcR293-CD27 and EcR293-CD40 cell lines were generated by their stable transfection with cDNAs for human CD27 and CD40, respectively. EcR293-CD27 cell lines expressing SIVA2 (EcR293-CD27-SIVA2) or SIVA1 (EcR293-CD27-SIVA1) were generated by transfection using the calcium phosphate method, and myc NIK and myc NIK (K670A) were later introduced into these cells by retroviral transduction and selection with 1 μg/ml puromycin. Ramos cells constitutively expressing myc-NIK were generated by retroviral transduction, followed by selection with 1 μg/ml puromycin. BJAB cells stably expressing myc-NIK were generated by electroporation and selection with 0.5 mg/ml G418. Later, SIVA2 was introduced into these cells by retroviral transduction and selection with 1 μg/ml puromycin. Ramos T-REx cells stably expressing the Tet repressor (Invitrogen) under blasticidin selection were generated using pcDNA6/TR plasmid and Amaxa nucleofection. These cells were transduced with SIVA2 (Ramos T-REx-SIVA2), SIVA2 (C73A) (Ramos T-REx-SIVA2 (C73A)), or SIVA1 (Ramos T-REx-SIVA1) cDNAs under the tetracyline operator and CMV promoter of pcDNA4 vector (Invitrogen) by the lentiviral system as described (Lois et al., 2002; Ramakrishnan et al., 2004). The T-REx cells were cultured in tetracycline-free serum (Invitrogen). SIVA1 and SIVA2 were induced with ponasterone (5 μg/ml) in EcR293 cells and with doxycyline (1 μg/ml) in Ramos T-REx cells.

Yeast two-hybrid tests. The cDNAs of NIK, SIVA, and TRAF2 were expressed in pGBKT7 or pGBT9 as bait and pGADT7 as prey vector. Binding was assayed in a SFY526 reporter yeast strain according to the instructions of the supplier (Clontech).

Mammalian expression vectors. SIVA2, SIVA1 were cloned from ESTs by PCR. The SIVA sequences were verified with the NCBI sequences NM_{—}006427 (SIVA1, SEQ ID NO. 10) and NM_{—}021709 (SIVA2 SEQ ID NO:11). The expression vectors for the extracellular domains of mCD70 and hCD40L, for myc-tagged wild-type and ‘kinase-dead’ NIK (KD-NIK), and for human CD27 have been previously described (Ramakrishnan, 2004). For retroviral transduction, myc-NIK were cloned into pBABE-5puro vector. pEGFP was purchased from Clontech. Point mutations in SIVA2, TRAF2, NIK, and Ubc13 were generated by site-directed mutagenesis using Pfu turbo DNA polymerase (Stratagene). FLAG-GST-BR3-ICD* (the intracellular domain of the BAFF receptor [amino acids 100-184] with a mutation [PVPAT>AVAAA] that prevents the binding of TRAF3 to it) was expressed in the same vector as that used for the expression 10 of the extracellular domains of mCD70 and hCD40L, except that in the latter case the leader sequence was removed. The cDNAs for the ubiquitin mutants have been described (Kovalenko et al., 2003). Enhanced green fluorescent protein plasmid (pEGFP) was purchased from Clontech. N-terminally FLAG-tagged cIAP1 and cIAP1 H588A (cIAP1 mut) were generated by subcloning from cIAP expression vectors, kindly provided by Dr. 15Gerry M. Cohen, University of Leicester.

Oligonucleotide sequences used for suppression of protein synthesis by RNA interference. The following siRNA sequences were introduced into the pSUPER vector (Brummelkamp et al., 2002), with the sequence ttcaagaga (SEQ ID NO. 1) used as a spacer: for human SIVA-NC3, sense strand 5% 20gatcccctgaataaacctctttatatttcaagagaatataaagaggtttattcatttttggaaa-3′(SEQ ID NO. 2) and antisense strand 5′-agatttccaaaaatgaataaacctattatattctcttgaaatataaagaggtttattcaggg-3′(SEQ ID NO. 3); for SIVA275, sense strand 5′-gatccccactgcagtgacatgtacgattcaagagatcgtacatgtcactgcagttttttggaaa-3′(SEQ ID NO. 4) and antisense strand 5′-agcttttccaaaaaactgcagtgacatgtacgatctcttgaatcgtacatgtcactgcagtggg-253′(SEQ ID NO. 5); for GFP, sense strand 5′-gatccccgctacctgttccatggccattcaagagatggccatggaacaggtagctttttggaaa-3′(SEQ ID NO. 6) and antisense strand 5′-agcttttccaaaaagctacctgaccatggccatctcttgaatggccatggaacaggtagcggg-3′(SEQ ID NO. 7).

Antibodies. A monoclonal antibody against human SIVA2 was raised in mice by their immunization with bacterially produced GST-SIVA2 and was affinity-purified with Trx-HIS-SIVA2. This antibody recognized both SIVA1 and SIVA2. Anti-HIS, anti-FLAG, anti-FLAG M2-beads, and anti-β-actin were purchased from Sigma. Anti-ubiquitin and anti-GST were from Covance, anti-GFP was purchased from Roche, anti CD27 CD27, TNFR1, TRAF2, Oct-1, and HA was purchased from Santa Cruz Biotechnology. The anti-NIK monoclonal antibody has been previously described {Ramakrishnan, 2004}. The anti-HA monoclonal antibody that was used for western analysis (clone-12CA5) and anti-myc monoclonal antibody (clone-9E10) were purified from mouse ascitic fluids on affinity columns to which their corresponding peptides were coupled.

Expression of recombinant proteins. For bacterial expression, GST-fusion proteins of SIVA2 were cloned into pGEX2T vector and expressed, according to the GST Gene Fusion System protocol of the manufacturer (Pharmacia Biotech). Transient transfections, total protein extractions, nuclear and cytoplasmic protein separations, immunoprecipitations, immunoblotting, and in-vitro kinase assays were carried out as described (Ramakrishnan et al., 2004). FLAG-SIVA2 was expressed using the pET44 vector, and TRAF3 (Trx-HIS-TRAF3) and SIVA2 (Trx-HIS-SIVA2) were expressed as Trx fusions using the pET32 vector (Novagen) in BL-21(DE3)pLysS cells (Novagen). Induction of all proteins was carried out at OD 600 of 0.4-0.5 with 0.2 mM isopropyl-β-D-thio-galactopyrano. Luciferase assay HEK293T cells (2×10⁵ cells) were seeded in 6-well plates and transfected by the calcium-phosphate precipitation method. Luciferase cDNA under control of the human immunodeficiency virus long terminal repeat (HIV-LTR) NF-κB promoter was used as the reporter plasmid. At the indicated times the cells were lysed in 120 μl of lysis buffer as described (Ausubel et al., 1996), and lysates of 10-20 μl were used for the assay with D-luciferin substrate in a Lumac Biocounter side for 4 h at 25° C.

In-vitro protein-binding assays The purified proteins were incubated at 30° C. for 1 h in 50 μl of buffer containing 30 mM HEPES pH 7.6, 5 mM MgCl₂, 150 mM NaCl, and 0.5 mM dithiothreitol (DTT). The binding mixture was later diluted to 1 ml in the same buffer plus 1% Triton X-100 and 1 mM EDTA, and was then subjected to immunoprecipitation.

siRNA and lentiviral transduction TRAF2, cIAP1, and siCONTROL nontargeting siRNAs were purchased from Dharmacon. siRNA was stably expressed by lentiviral transduction as previously described (Ramakrishnan et al., 2004). siRNAs were transiently transfected with Lipofectamine 2000 reagent (Invitrogen).

In-vitro ubiquitination. Ubiquitination in vitro was assayed in a 50-μl reaction volume containing recombinant ubiquitin (8 μg), E1 enzyme (0.2 μg), the indicated E2 enzyme (0.5 μg), and 1-2 μg of recombinant GST-SIVA2 or GST-SIVA2 (C73A) bound to glutathione agarose in a buffer containing 30 mM HEPES pH 7.6, 5 mM MgCl₂, 2 mM ATP, 0.5 mM DTT, 10 mM sodium citrate, 10 mM creatine phosphate, 0.2 μg/ml creatine kinase and 5 μM ubiquitin aldehyde. Reactions were incubated at 37° C. for 2 h with intermittent agitation. Supernatants of the reaction were analyzed for free polyubiquitin chains formed in solution. For assay of self-ubiquitination of SIVA2 the glutathione beads were washed three times with a buffer containing 20 mM HEPES pH 7.6, 250 mM NaCl, 1 mM DTT, 1% Triton X-100 and Complete Protease Inhibitor Cocktail, boiled with LDS sample buffer (Invitrogen), and analyzed by western blotting. For the in-vitro substrate ubiquitination assay, 0.5-1 μg of recombinant GST-TRAF2, Trx-HIS-TRAF3, cellular TRAF2 (C34A), or cIAP1 was added in the reactions. After incubation, reactions were terminated by boiling in sample buffer with 1% sodium dodecyl sulfate (SDS). The boiled samples were diluted to 1 ml with buffer containing 20 mM HEPES pH 7.6, 150 mM NaCl, 0.2% NP-40, 1 mM EDTA, and Complete Protease Inhibitor Cocktail. Specific proteins were immunoprecipitated for 2 h at 4° C. and assayed by western blotting, using 4-12% NuPAGE Novex Bis-Tris gels (Invitrogen).

Semiquantitative RT-PCR and real-time PCR. RNA was prepared using the RNeasy Mini Kit (Qiagen) according to the manufacturer's instructions. Semiquantitative RT-PCR for SIVA2 message was performed with MMLV reverse transcriptase and oligo dT primer (Promega). SIVA1, SIVA2, and SIVA3 were following primers: sense strand 5′-cgcggatccaacatgcccaagcggagctgcccc-3′(SEQ ID NO. 8), which contains a BamHI site, and antisense strand 5′-ccgctcgaggccagcctcaggtctcgaacatgg-3′(SEQ ID NO. 9), which contains a XhoI site.

In vivo [³²P]orthophosphate labeling. Phosphorylation of SIVA2 in cells was assessed by metabolic labeling with [³²P]orthophosphate in HEK-293T cells. Cells were cultured, 22 h after transfection, in phosphate-free medium with 10% dialysed serum. Serum was dialysed against 10 mM tricine-buffered saline, pH 7.4, for 48 h. Following starvation for 90 min in phosphate-free medium, [³²P]orthophosphate (0.4 mCi/ml) was added for an additional 90-min period. MG132 was added to one sample for the last 2 h. Cells were harvested 25 h after transfection, washed twice with phosphate-free medium, and lysed in kinase lysis buffer (Ramakrishnan et al., 2004). SIVA2 was immunoprecipitated through the FLAG tag, and phosphate incorporation was assessed by autoradiography.

Electroelution of SIVA2 from Coomassie blue-stained gel. SIVA2 was isolated from extracts of HEK-293T cells cotransfected with FLAG-SIVA2 and NIK by immunoprecipitation with anti-FLAG-M2 beads and, following SDS-PAGE, was electroeluted in a GeBAflex-tube (Gene Bio Application) at 150 V for 2 h. The elution buffer contained 0.025% (w/v) SDS, 25 mM Tris buffer, and 250 mM Tricine buffer (pH 8.5). Following electroelution, SDS was removed by precipitation in cold 50% (w/v) trichloroacetic acid in the presence of 0.5% (w/v) sodium deoxycholate (Montigny, C., et al. Fe2+-catalyzed oxidative cleavages of Ca2+-ATPase reveal novel features of its pumping mechanism. J Biol Chem 279, 43971-43981 (2004)), and the samples were analyzed by mass spectrometry (MS).

In-gel digestion. Protein bands were excised from the SDS gel, stained with Gel Code, and destained by multiple washings with 50% acetonitrile in 50 mM ammonium bicarbonate. The protein bands were subsequently reduced, alkylated, and subjection to in-gel digestion by bovine trypsin (sequencing grade, Roche), Lys C, and endoproteinase Glu-C (V8) (both from Boehringer Mannheim) at a concentration of 12.5 ng/μl in 50 mM ammonium bicarbonate at 37° C., as described (Shevchenko, A., Wilm, M., Vorm, O. & Mann, M. Mass spectrometric sequencing of proteins silver-stained polyacrylamide gels. Anal Chem 68, 850-858 (1996)). The extracted peptide solution was dried for subsequent Matrix-Assisted Laser Desorption Time-Of Flight-ionization (MALDI-TOF) and electrospray ionization-mass spectrometric (ESI-MS) analyses.

Sample preparation. Aliquots of the extracted peptide mixture dissolved in 0.1% trifluoroacetic acid were used for MALDI-TOF MS by means of either fast evaporation (Jensen, O. N., Podtelejnikov, A. & Mann, M. Delayed extraction improves specificity in database searches by matrix-assisted laser desorption/ionization peptide maps. Rapid Commun Mass Spectrom 10, 1371-1378 (1996)) or dry droplet (Kussmann, M., Lassing, U., Sturmer, C. A., Przybylski, M. & Roepstorff, P. Matrix-assisted laser desorption/ionization mass spectrometric peptide mapping of the neural cell adhesion protein neurolin purified by sodium dodecyl sulfate polyacrylamide gel electrophoresis or acidic precipitation. J Mass Spectrom 32, 483-493 (1997)) methods. α-Cyano-4-hydroxy-cinnamic acid (HCCA) or 2,5-dihydroxybenzoic acid or both were used as matrixes for analysis. Samples were purified and prepared for ESI-MS as described previously (Wilm, M., Neubauer, G. & Mann, M. Parent ion scans of unseparated peptide mixtures. Anal Chem 68, 527-533 (1996). Microcolumns were prepared with R2 reverse phase material (PerSeptive Biosystem, Framingham). Peptides were eluted with 60% methanol/5% formic acid directly into a nano-electrospray capillary.

Intact mass measurement. This was done with a Reflex III MALDI-TOF mass spectrometer (Bruker) equipped with a delayed extraction ion source, a reflector, and a 337-nm nitrogen laser. Electroeluted protein was dissolved in 1-2 μl of 80% formic acid and immediately diluted with MilliQ H₂O to a final concentration of 10%. Samples were sonicated for 5-10 min at 25° C. Part of the sample (5%-25%) was used for the analysis. DHB was used as a matrix.

Protein identification by peptide mass mapping and nano-liquid chromatography-tandem mass spectrometry (nano-LC-ESI-MS/MS). These procedures were performed with a Reflex III MALDI-TOF mass spectrometer and an API Q-STAR Pulsarⁱ Electrospray-Quadrupole TOF tandem mass spectrometer with a quadrupole collision cell (MDS-Sciex) equipped with a nano-electrospray source (MDS Proteomics).

Precursor ion scan and nano-ESI-MS/MS. Precursor ion scan experiments for specific detection of phosphorylated serine, threonine, and tyrosine amino-acid residues at m/z of −67, −79 and −97 (Neubauer, G. & Mann, M. Mapping of phosphorylation sites of gel-isolated proteins by nanoelectrospray tandem mass spectrometry: potentials and limitations. Anal Chem 71, 235-242 (1999)), and MS/MS sequencing of the phosphopeptides were performed on an API Q-STAR Pulsarⁱ Electrospray-Quadrupole TOF. Mass resolution was routinely obtained in the range of 10,000 to 15,000 (for both conventional mass spectrometric and MS/MS modes of operation), and a mass measurement accuracy of at least 0.02 Da with external calibration was achieved. Approximately 2 μl of sample was loaded into a nanoelectrospray tip. For precursor ion-scan experiments the peptide mixture was desalted using a double alignment of desalting capillaries filled with Poros R2 and Poros oligoR3 sorbent (PerSeptive Biosystems) prepared and operated essentially as described⁶. For identification of phosphorylation sites, the data on all the peptides that had accumulated during the multiple precursor ion-scan experiments in negative mode were first analyzed and compared to ESI-MSTOF spectra (recorded in positive ion mode, to assign the charge states of the peptides. The peptides that were hypothesized based on precursor ion scan experiments (Kalkum, M., Lyon, G. J. & Chait, B. T. Detection of secreted peptides by using hypothesis-driven multistage mass spectrometry. Proc Natl Acad Sci USA 100, 2795-2800 (2003)) were subjected to further analysis by nano-ESI-MS/MS.

Nano-LC-ESI-MS/MS. This was carried out with a nano-liquid chromatography system incorporating the Ultimate Capillary/Nano LC System, consisting of a FAMOS Micro Autosampler and a Switchos Micro-Column Switching Module (LC Packings, Dionex) on line with an API Q-STAR Pulsarⁱ Electrospray-Quadrupole TOF tandem mass spectrometer. A C₁₈ nanocolumn (internal diameter (i.d.) 75 μm, length 15 mm, particle size 5 μM (LC Packings, Dionex)) was used. Flow rate through the column was 150 nl/min. A methanol-acetonitrile gradient with a mobile phase containing 0.1% and 2% formic acid in buffers A and B, respectively, was employed. The gradient used was 5%-50% acetonitrile over 45 min. The injection volume was 5 μl. In the nano-electrospray ionization source, the end of the capillary from the nano-LC column was connected to the emitter with pico-tip silica tubing, i.d. 20 μm (New Objective), by a stainless steel union, with a PEEK sleeve for coupling the nanospray with the on-line nano-LC. To produce an electrospray the voltage applied to the union in was 2 kV, and the cone voltage was 3 V. Argon was introduced as a collision gas at a pressure of 1 psi. The peptides retrieved by nano-ESI-MS/MS and nano-LC-ESI-MS/MS were identified, and the location of their phosphorylated residues was determined from the detected collision-induced dissociation products by Mascot software (Matrix Science), and confirmed by manual inspection of the fragmentation series.

Metabolic glycoprotein labeling. In vivo O-GlcNAcylation of SIVA2 co-expressed with NIK was detected with the Click-iT GlcNAz metabolic glycoprotein labeling reagent and Biotin Glycoprotein Detection Kit (Invitrogen) according to the manufacturer's instructions. Labeled proteins were detected by western blotting with streptavidin HRP.

Wheat-germ agglutinin-lectin binding assay. Cells were lysed in a buffer containing 50 mM HEPES pH 7.6, 150 mM NaCl, 1% Triton X-100, and EDTA-free Complete Protease Inhibitor Cocktail (Roche). WGA agarose beads were washed twice with lysis buffer and added to the lysates. Binding of glycoproteins to the lectin was allowed to proceed at 4° C. for 2 h in a rotator. To check the specificity of O-GlcNAcylated SIVA2 binding to WGA, 0.5 M N-acetyl-D-glucosamine was added as a competitor in the lectin-binding assay. After incubation, the WGA beads were washed three times with the lysis buffer and bound SIVA2 was analyzed by western blotting.

### Example 1

### Cytokine-Induced Stabilization of SIVA2

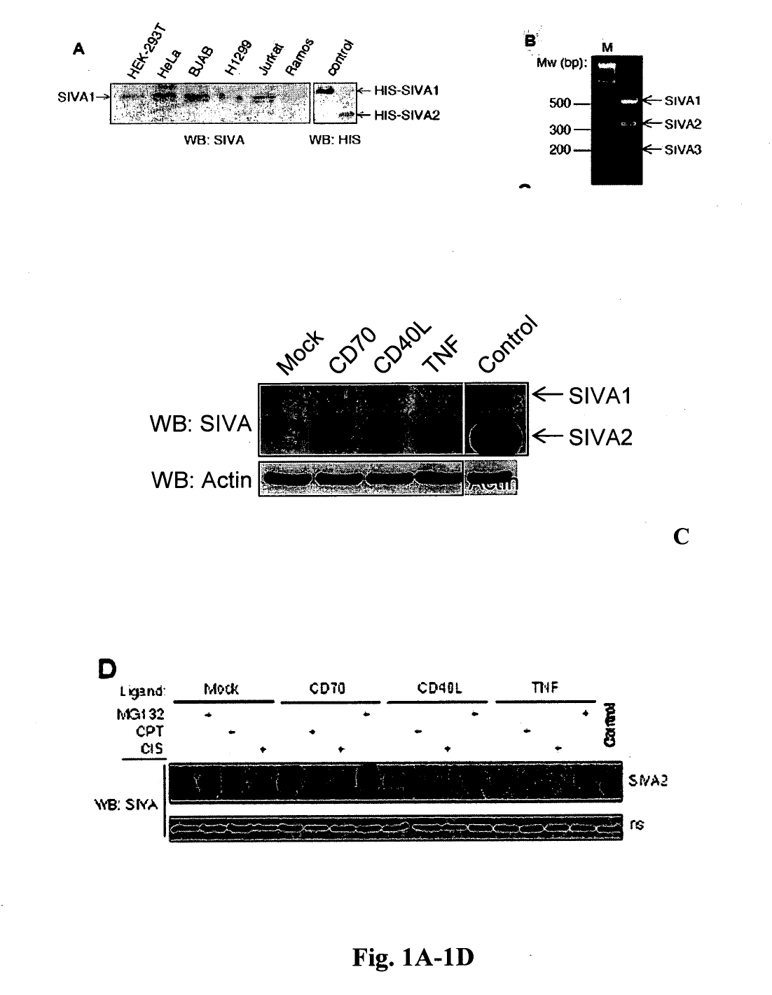

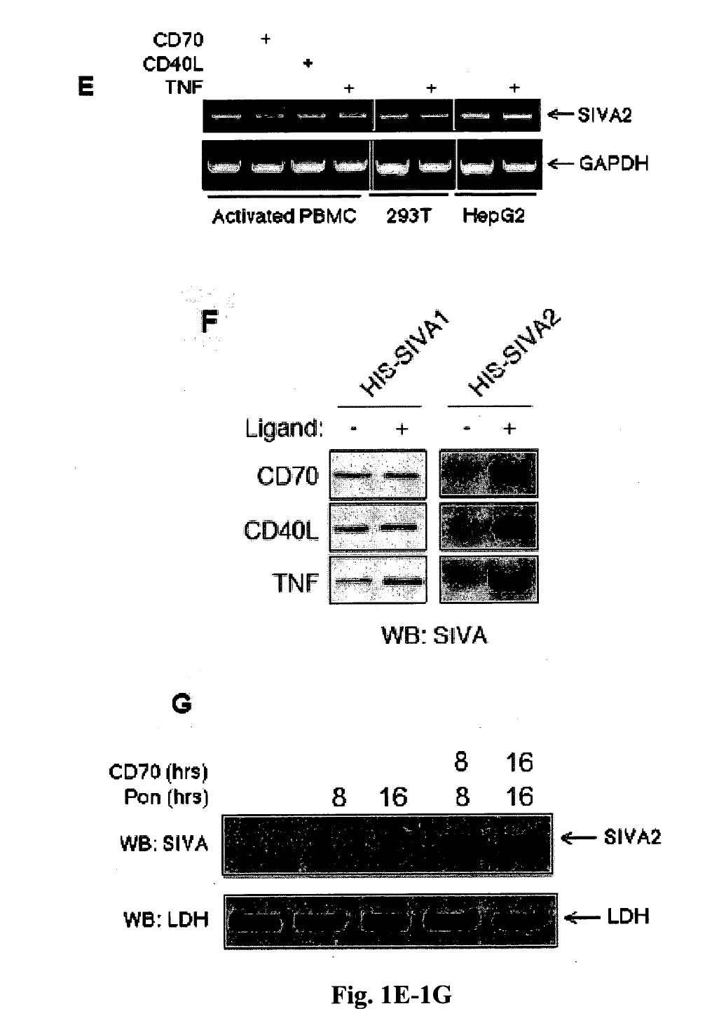

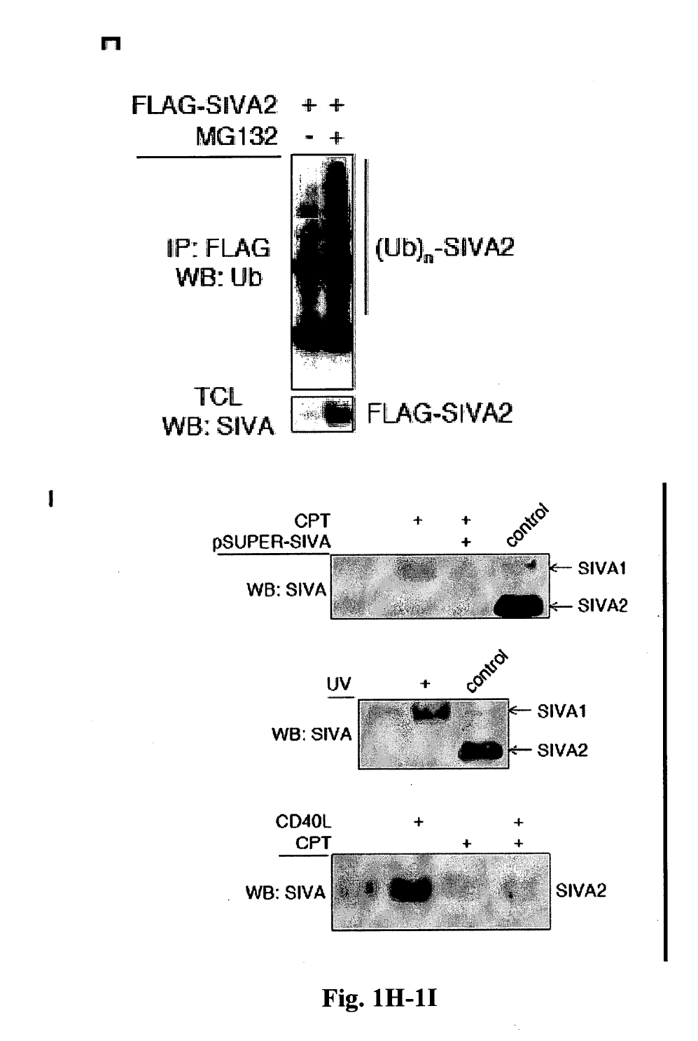

Although many cell types express the mRNAs for both SIVA1 and SIVA2, in our examination of various cell lines we were able to detect significant amounts of the SIVA1 protein only (FIG. 1A, left panel). Moreover, in transient-transfection experiments the SIVA2 cDNA was poorly expressed compared to SIVA1 (FIG. 1A, right panel).

On closer examination of SIVA expression in peripheral-blood mononuclear cells (PBMCs) transcripts of both splice variants were found, as well of a yet shorter variant (‘SIVA3’) corresponding to exons 1 and 4, but very little of the proteins themselves (FIG. 1B and data not shown). However, treatment of the cells with CD70 (CD27 ligand), CD154 (CD40 ligand, CD40L), or TNF, resulted in extensive enhancement of SIVA2 expression but did not affect expression of SIVA1 (FIG. 1C). An increase restricted to SIVA2 was also observed in cells treated by these cytokines following pre-activation with phytohemagglutinin (PHA). The apparent molecular size of SIVA2 in the pre-activated cells, however, was somewhat larger than that of the protein produced by transfection of cells with the SIVA2 cDNA, probably as a consequence of some post-translational modification(s) of the protein (FIG. 1D). This increase in the SIVA2 protein was not associated with any change in its transcript level (FIG. 1E), suggesting that it occurs post-transcriptionally. The above three ligands of the TNF family were also found to enhance the expression of SIVA2, but not of SIVA1, in cells transfected with the corresponding cDNA (FIG. 1F) as well in cells that expressed constitutively cDNA constructs allowing inducible expression of either SIVA1 or SIVA2 (FIG. 1G and data not shown). Blocking proteasomal function also caused a dramatic enhancement in the expression of SIVA2, both in PBMCs (FIG. 1D) and in transfected cell lines (FIG. 1H). It also increased the accumulation of ubiquitinated forms of the protein (FIG. 1H). Application of genotoxic agents and oxidative stress, which enhance SIVA1 expression (Xue, 2002; Padanilam, 1998; Qin, 2002; Daoud, 2003; Fortin, 2004; Jacobs, 2007) and FIG. 1I, top and middle panels), did not enhance the expression of SIVA2 and in fact antagonized its enhancement by cytokines (FIG. 1D, and FIG. 1I, bottom panel).

These findings suggested that ligands of the TNF family have the ability to stabilize the SIVA2 protein.

### Example 2

### TRAF2 and NIK, Independently, Contribute to Ligand-Induced Stabilization of SIVA2, While cIAP1 Facilitates SIVA2 Degradation

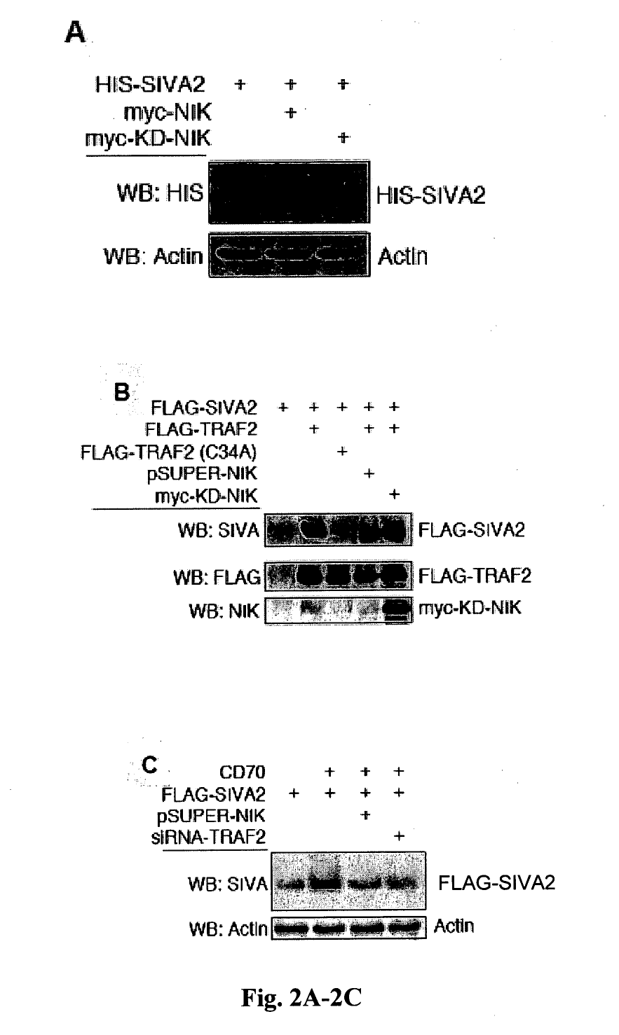

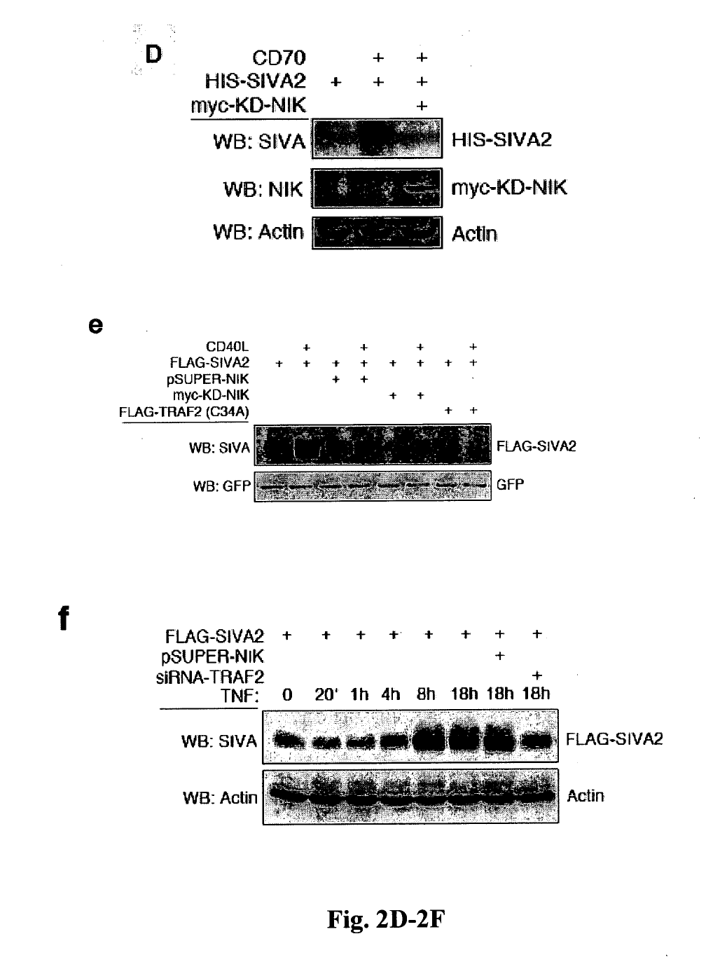

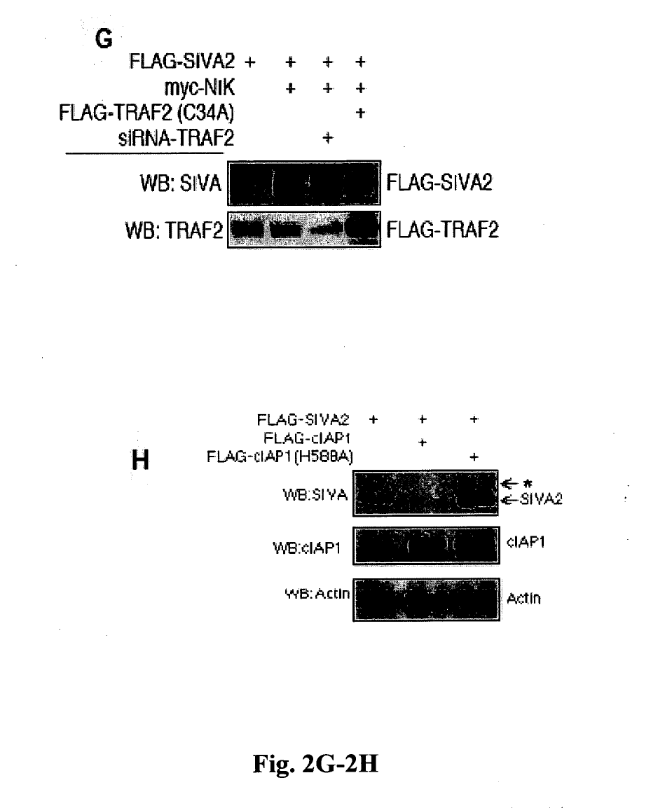

SIVA2 is recruited to the signaling complexes of several receptors of the TNF family and it was found to bind specifically to three signaling proteins that these receptors employ: the ubiquitin ligases TRAF2 and cIAP1 (see Examples below) and the protein kinase NIK ({Ramakrishnan, 2004} and Ramakrishnan et al., submitted). It was found by assessing the impact of these signaling proteins on SIVA2 that expression of this protein was dramatically upregulated when it was co-expressed with NIK (FIG. 2A), but not with the enzymatically inactive NIK mutant, KD-NIK (FIG. 2A). It was also strongly upregulated when co-expressed with TRAF2 (FIG. 2B). Yet not with TRAF2 (C34A), a TRAF2 mutant deficient in ubiquitin-ligase activity (FIG. 2B), suggesting that NIK and TRAF2 activity contribute to its ligand-induced stabilization. In line with this notion, stabilization of SIVA2 by CD70 (FIG. 2C, D) or CD40L (FIG. 2E) was drastically reduced by interference with the function or expression of either NIK or TRAF2; moreover, its stabilization by TNF, whose signaling activity does not employ NIK and does not induce its association with SIVA2 was decreased only by interference with the function of TRAF2 (FIG. 2F).

Overexpression of the TRAF2 (C34A) mutant or transfection of the cells with TRAF2 siRNA had no effect on NIK-induced stabilization of SIVA2 (FIG. 2G), nor did overexpression of KD-NIK or knockdown of NIK expression interfere with SIVA2 stabilization by TRAF2 (FIG. 2B). These findings suggested that the two proteins enhance SIVA2 stability by mechanisms that are, at least partly, distinct.

Testing further the impact of cIAP1 binding on SIVA2 expression, it was found that over-expression of cIAP1 resulted in dramatic reduction in the amount of transiently expressed SIVA2 (FIG. 2H). In contrast, over-expression of a ring-finger mutant of cIAP1 (H588A) that is devoid of ubiquitin-ligase activity enhanced SIVA2 expression. It also facilitated accumulation of a modified form of the protein with a higher apparent molecular size (arrow in FIG. 2H). However siRNA-mediated knockdown of cIAP1 had no effect on SIVA2 expression (data not shown). These findings suggested that, although cIAP1 may in some situations facilitate SIVA2 degradation, not this ubiquitin ligase but another one is responsible for the low stability of SIVA2 in our tested cells. The ability of the H588A mutant of cIAP1 to enhance SIVA2 expression may reflect competition between cIAP1 and that other, yet unknown, ubiquitin ligase for a common binding site on SIVA2.

### Example 3

### SIVA is O-GlcNAcylated, and this Modification Seems to Contribute to SIVA2 Stabilization by NIK and TRAF2

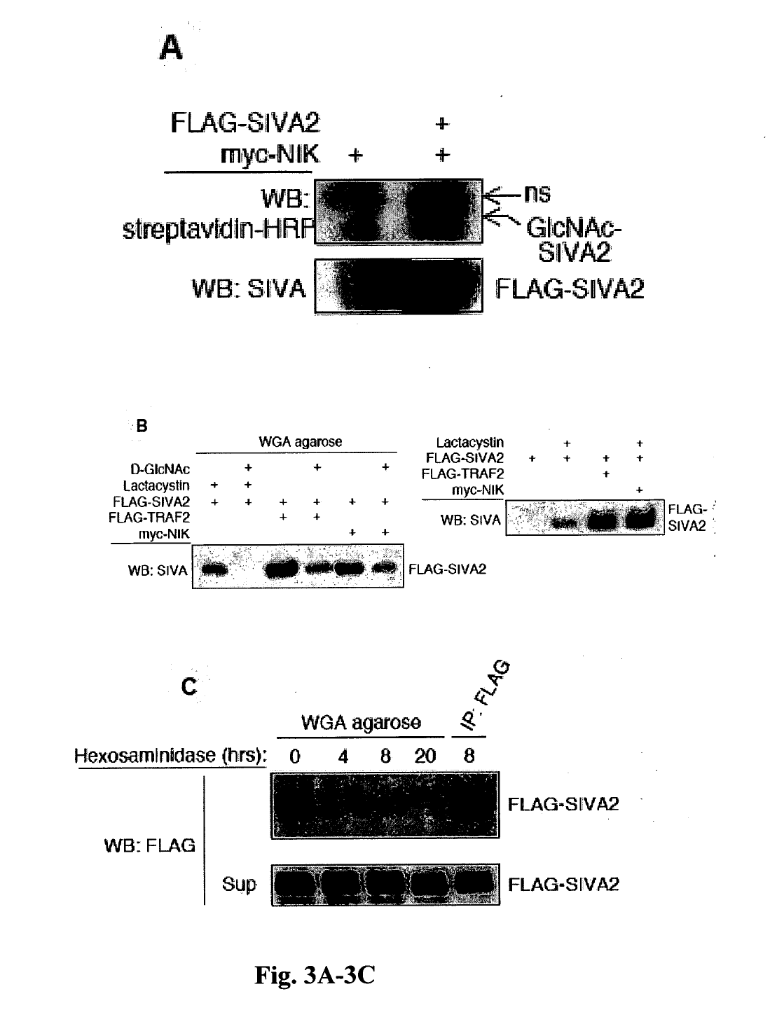

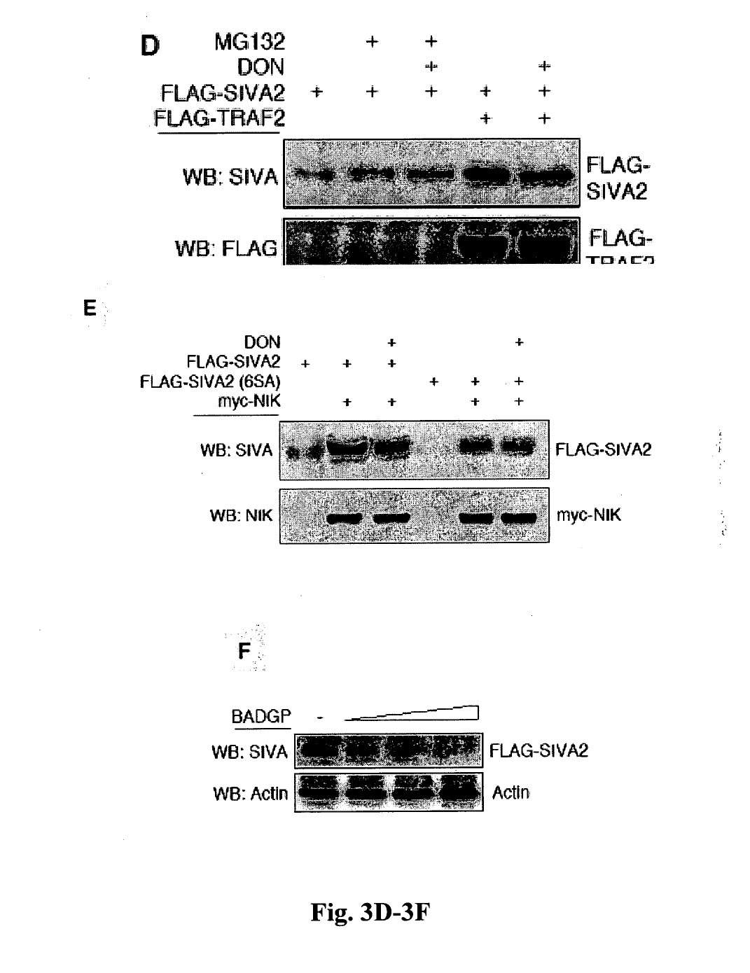

Modulation of protein stability can be induced by various kinds of covalent modifications, including serine, threonine, or tyrosine phosphorylation, O-linked N-acetylglucosamine modification (O-GlcNAcylation) of serine or threonine, and linkage of ubiquitin or one of its homologues, mostly to lysine residues. SIVA2 was found to occur in cells in O-linked N-acetylglucosamine modified forms, as assessed by in-vivo labeling (FIG. 3A), wheatgerm-agglutinin (WGA) binding (FIG. 3B), and β-D-N-acetyl hexosaminidase treatment (FIG. 3C). Inhibition of O-GlcNAcylation decreased the stabilization of SIVA2 by TRAF2 (FIG. 3D) or NIK (FIG. 3E, F), but not by a proteasomal inhibitor (FIG. 3D) suggesting that this modification is required for maintaining SIVA2 in a stable form.

### Example 4

### SIVA2 is Phosphorylated in Multiple Serine Residues at its N-Terminus and this Phosphorylation Seems Also to Contribute to its Stabilization

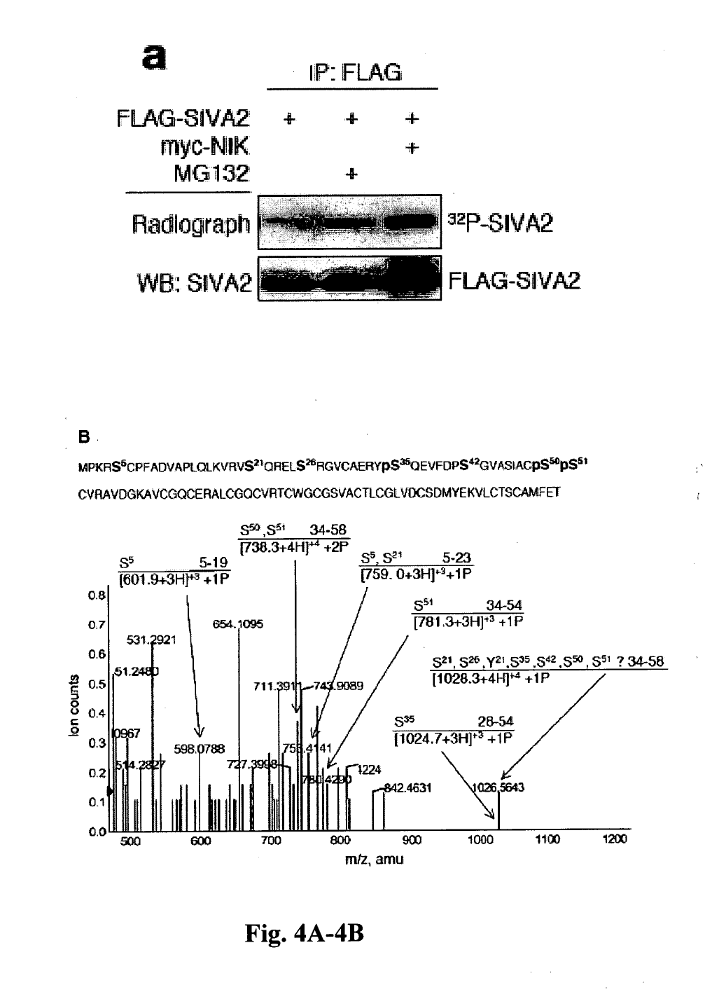

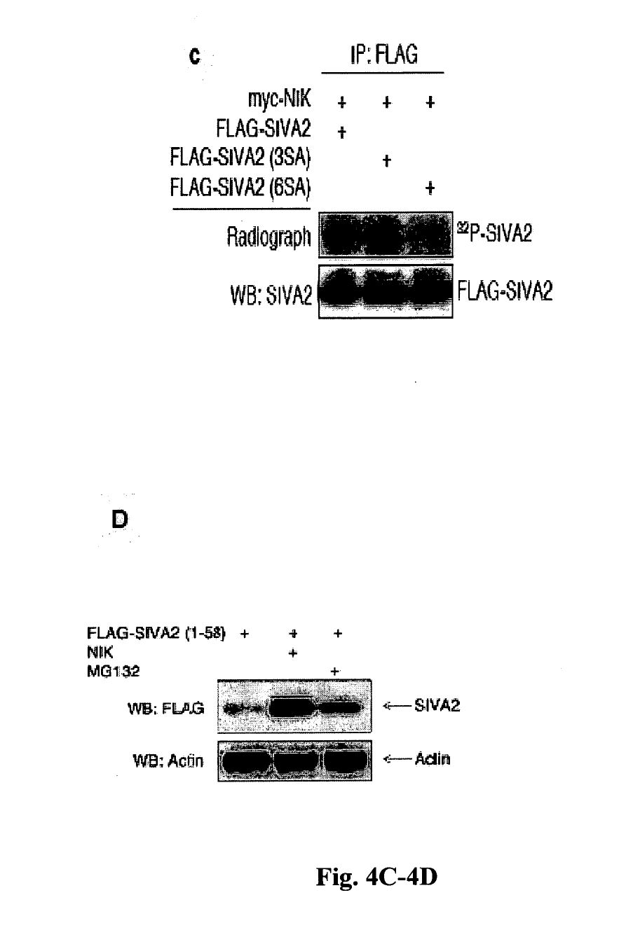

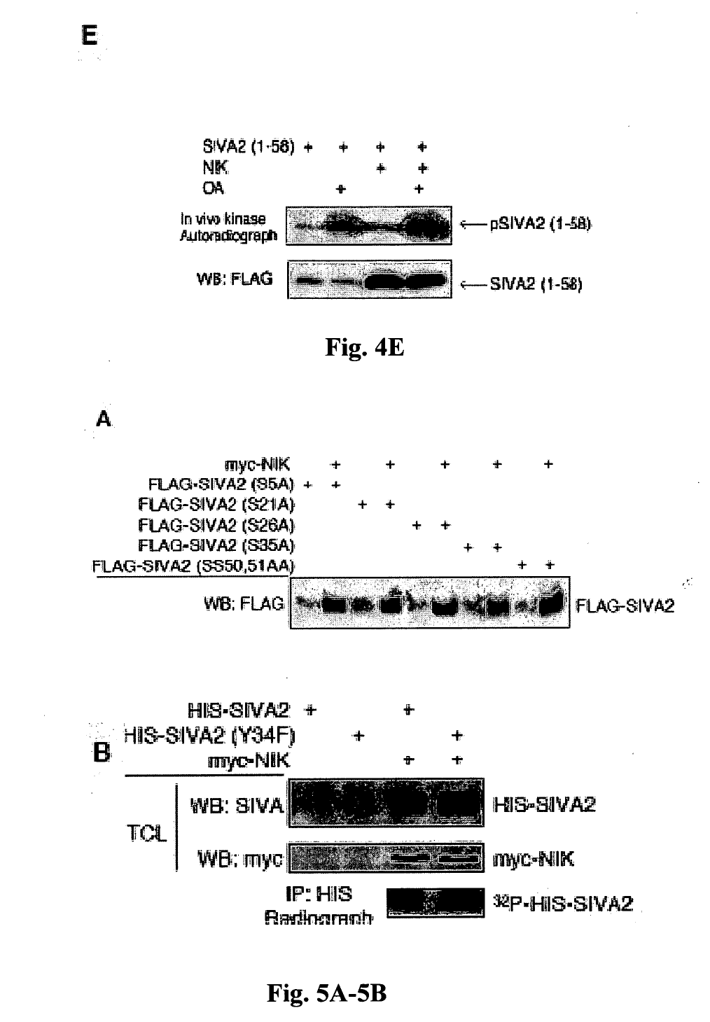

Assessment of ³²P incorporation into SIVA2 in cells cotransfected with NIK disclosed that SIVA2 is subject to phosphorylation (FIG. 4A). In mass-spectrometric (MS) analysis of SIVA2 isolated from cells cotransfected with NIK, phosphate was found to be linked to several serine residues at its N-terminus (FIG. 4B, Figs. S1 and S2 and Table S1), though not to the tyrosine at position 34, which was previously reported to be phosphorylated in response to oxidative stress {Cao, 2001}.

Since only enzymatically-active NIK stabilizes SIVA2 (FIG. 2A), and since NIK binds specifically to SIVA2, it seemed plausible that the stabilization of SIVA2 by NIK occurs as a consequence of its direct phosphorylation by the latter. Indeed, it was found that, when immuopurified from cells that over-express NIK, SIVA2 preparations were effectively phosphorylated in vitro by some associated protein kinase(s) (FIG. 4C). However, in deletion analysis of the NIK effect on SIVA2 it was found that NIK also enhances the phosphorylation and expression of SIVA2 (1-58), a deletion mutant of SIVA2 deficient of its C-terminal cysteine-rich region (CRR), which, as reported elsewhere, (Ramakrishnan et al. submitted) is the region in SIVA2 to which NIK binds (FIG. 4D, E). This finding suggested that SIVA2 stabilization by NIK does not require their direct association. A plausible explanation for these observations was that NIK enhances the activity of another SIVA2-associated protein kinase, which in turn phosphorylates the N-terminal part of SIVA2 and thus enhances its stability.

### Example 5

### Identification of Amino Acid Residues in SIVA2 that Contribute to its Stabilization by NIK and TRAF2

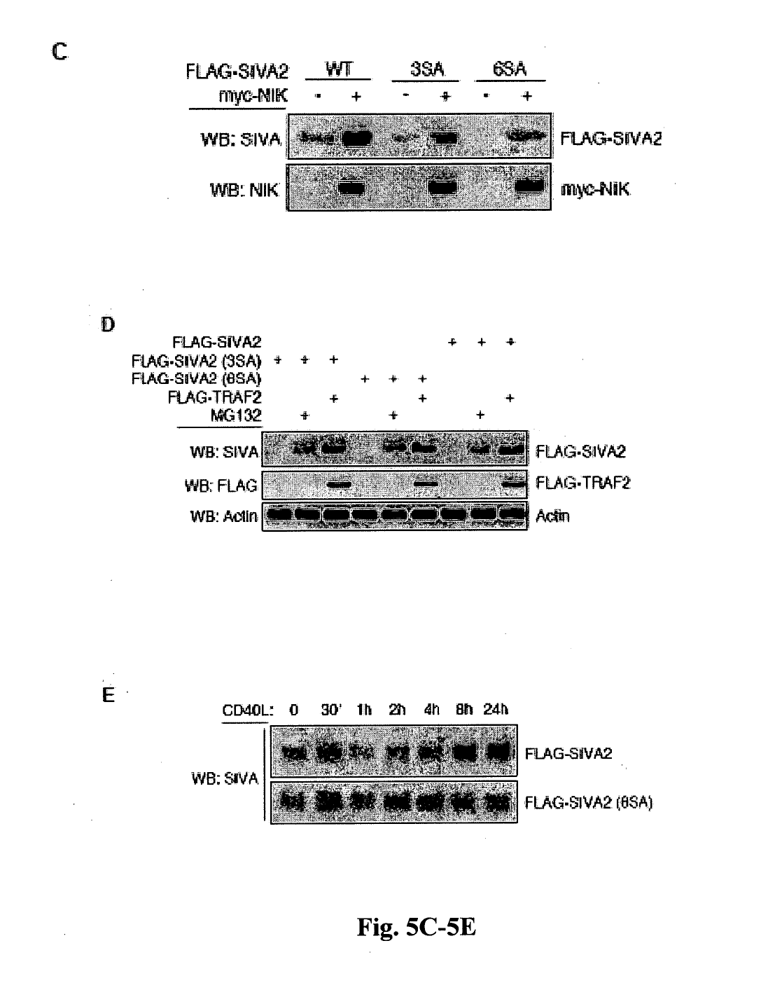

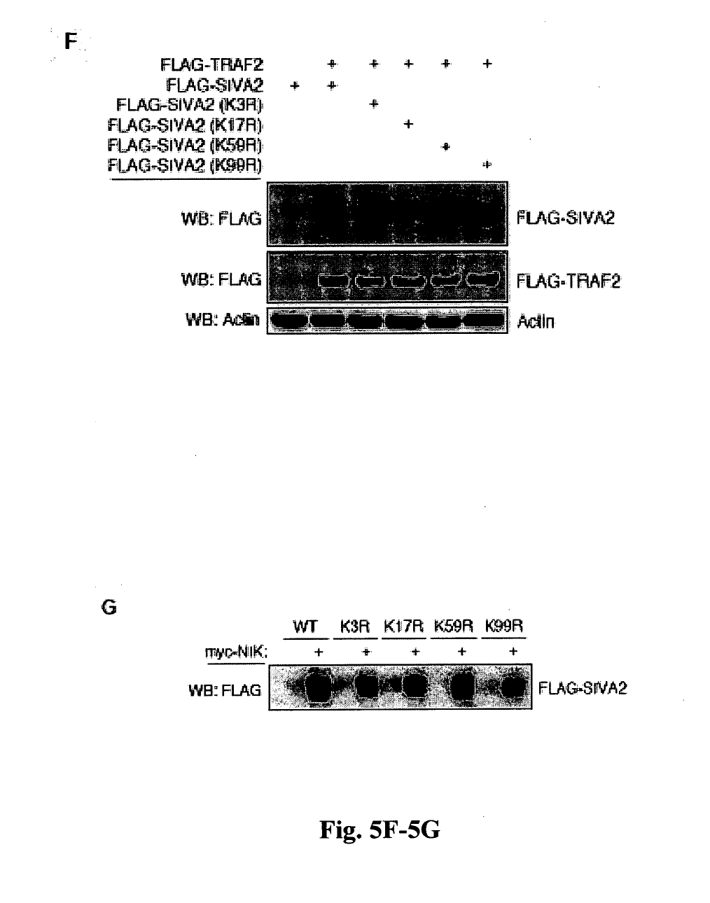

In view of the findings suggesting involvement of phosphorylation and glycosylation of SIVA2, as well as involvement of the region in TRAF2 which is required for protein ubiquitination by it, in the regulation of SIVA2 stability, it was assessed the impact of mutations of potential target sites in SIVA2 for these modifications on SIVA2 stability. Mutations of none of the individual serine residues that were found to be phosphorylated in SIVA2 had any effect on the extent of SIVA2 stabilization (FIG. 5A). Nor was SIVA2 stabilization by NIK found to be affected by mutation of tyrosine 34 (FIG. 5B). However, the combined mutations of three of the serine residues that were found to be phosphorylated (residues 5, 50, 51; ‘3SA’), and even more so of six (residues 5, 21, 26, 35, 50, 51; ‘6SA’), effectively decreased the phosphorylation of SIVA2 in vitro (FIG. 4C), and also decreased its stabilization by NIK (FIG. 5C), while still allowing it to be stabilized by proteasomal inhibitors (FIG. 5D). Unlike the wild-type protein, the 6SA mutant could not be stabilized by CD40L (FIG. 5E). However, consistently with our findings suggesting that TRAF2 and NIK stabilize SIVA2 by means of different mechanisms, mutation of the above six serines did not compromise the TRAF2-stabilizing effect (FIG. 5F).

Further examining the involvement of lysine residues in SIVA2 in its stabilization, it was found that mutation of either one of two of the residues, K17 and K99, abolished the protein's stabilization by TRAF2 (FIG. 50. These mutations did not affect, however, the stabilization of SIVA2 by NIK (FIG. 5G).

### Example 6

### SIVA2 Binds to NIK, Traf2 and cIAP

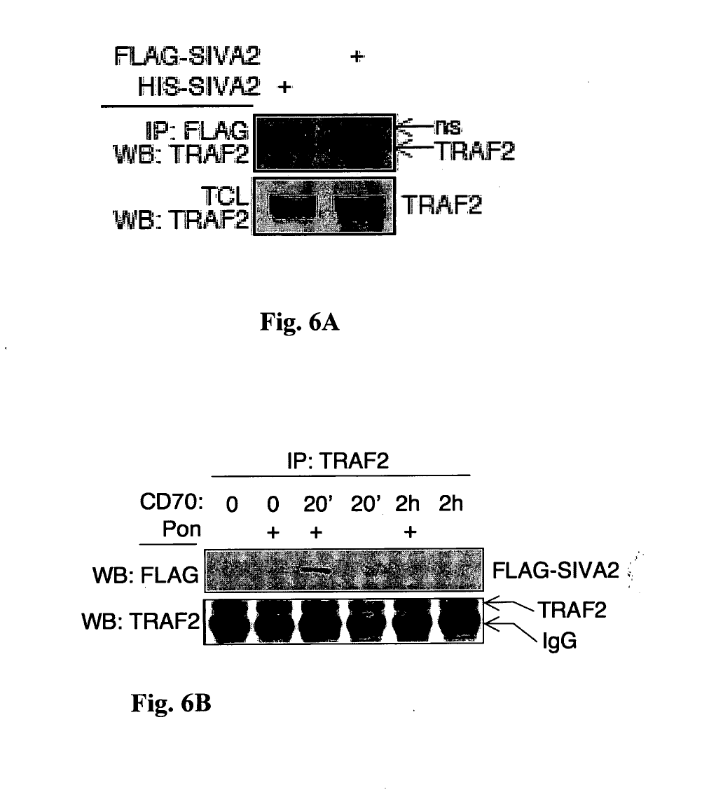

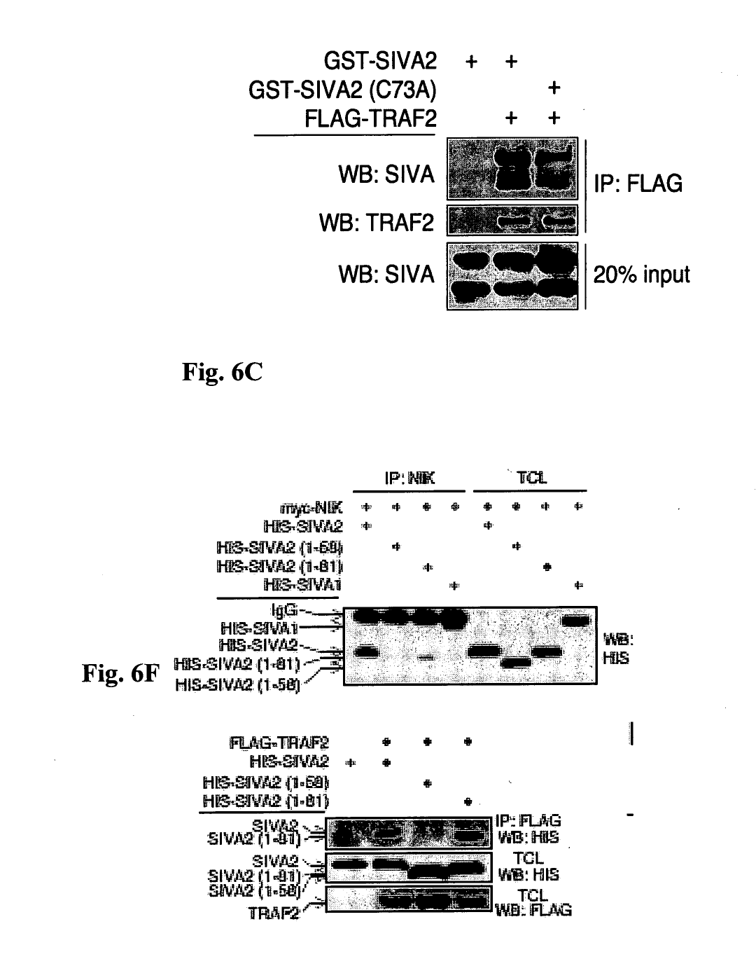

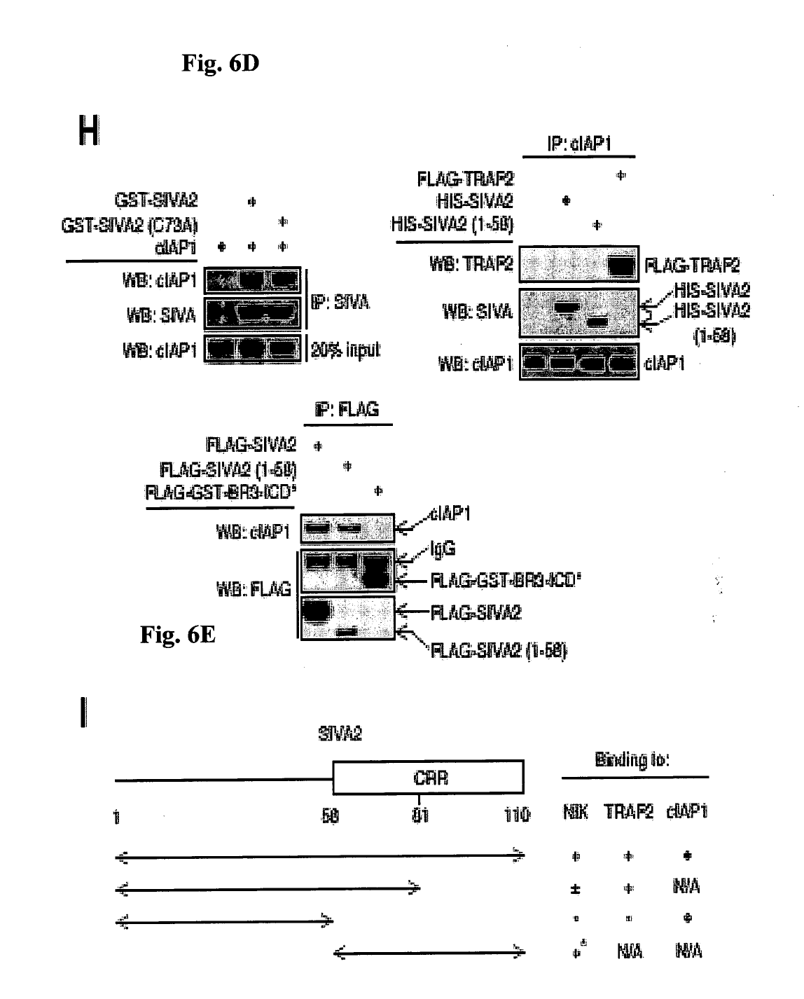

On examining the binding of SIVA2 to various other proteins known to mediate signaling by receptors of the TNF/NGF family, it was found that it also binds to TRAF2 (FIG. 6 (A-C) and cIAP1 FIG. 6(D), but not to TRAF3 (not shown). Deletion analysis suggested that TRAF2, like NIK binds to CRR in SIVA2 (FIG. 6 (F), lower panel). On the other hand cIAP1 was found to bind to the N-terminal part of SIVA2, upstream of the CRR FIG. 6 D right and bottom panels, and FIG. 6 (E).

### Example 7

### SIVA2 Inhibits TRAF2 and NIK Mediated Signaling

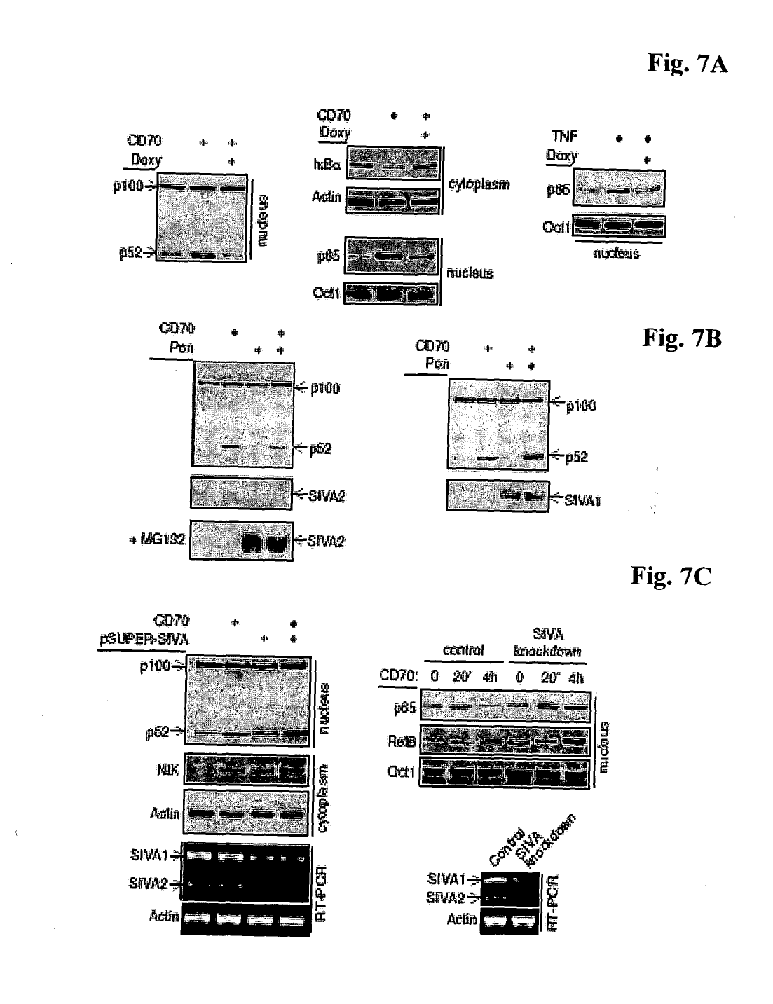

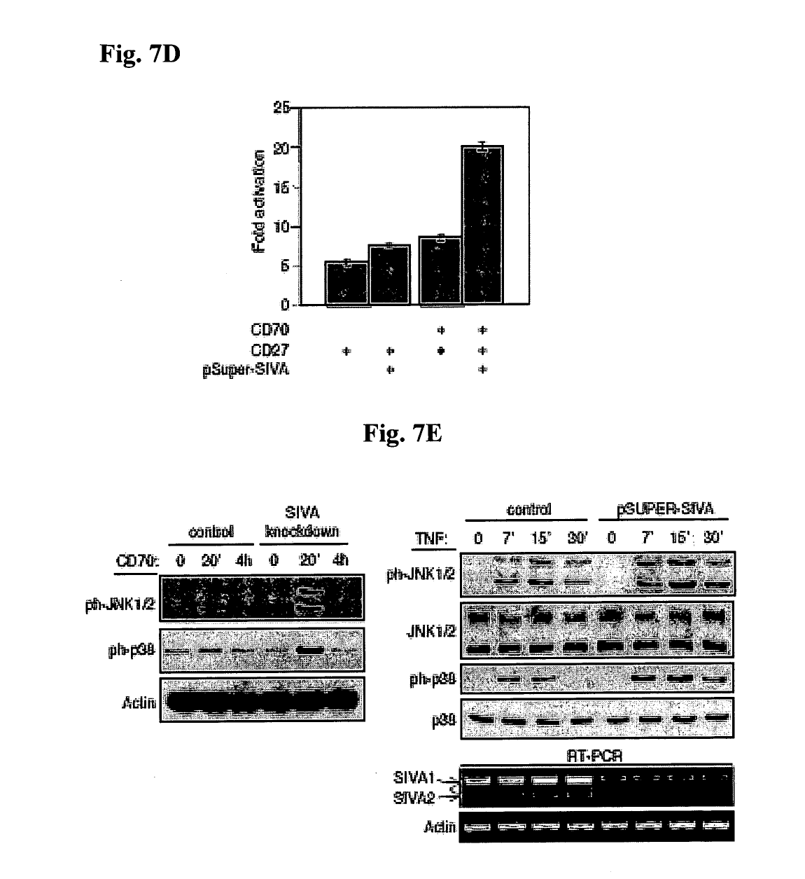

In assessing the functional significance of the protein associations described above, it was found that induction of SIVA2 suppresses the activation of both the alternative and the canonical NF-κB pathways by CD70 (FIG. 7A, left and middle panels, and FIG. 7B upper panels) as well as activation of the canonical pathway by TNF (FIG. 7A, right panel). SIVA1, on the other hand, although expressed at much higher level than SIVA2, had no such effect (FIG. 7B, right panel).

Conversely, cells in which SIVA expression has been knocked down displayed constitutive activation of the alternative NF-κB pathway (FIG. 7C, left panel). They also displayed somewhat increased basal levels of canonical NF-κB pathway and heightened responsiveness of this pathway to activation by CD70, as reflected both in the extent of translocation of p65 NF-κB protein to the nucleus (FIG. 7 (C, right panel) and luciferase reporter tests (FIG. 7D). Knockdown of SIVA also enhanced the induction of JNK and p38 kinase phosphorylation both by CD70 and by TNF (FIG. 7E)

### Example 8

### SIVA2, cooperatively with cIAP1, Mediates Ubiquitination and Degradation of TRAF2 in Response to CD27

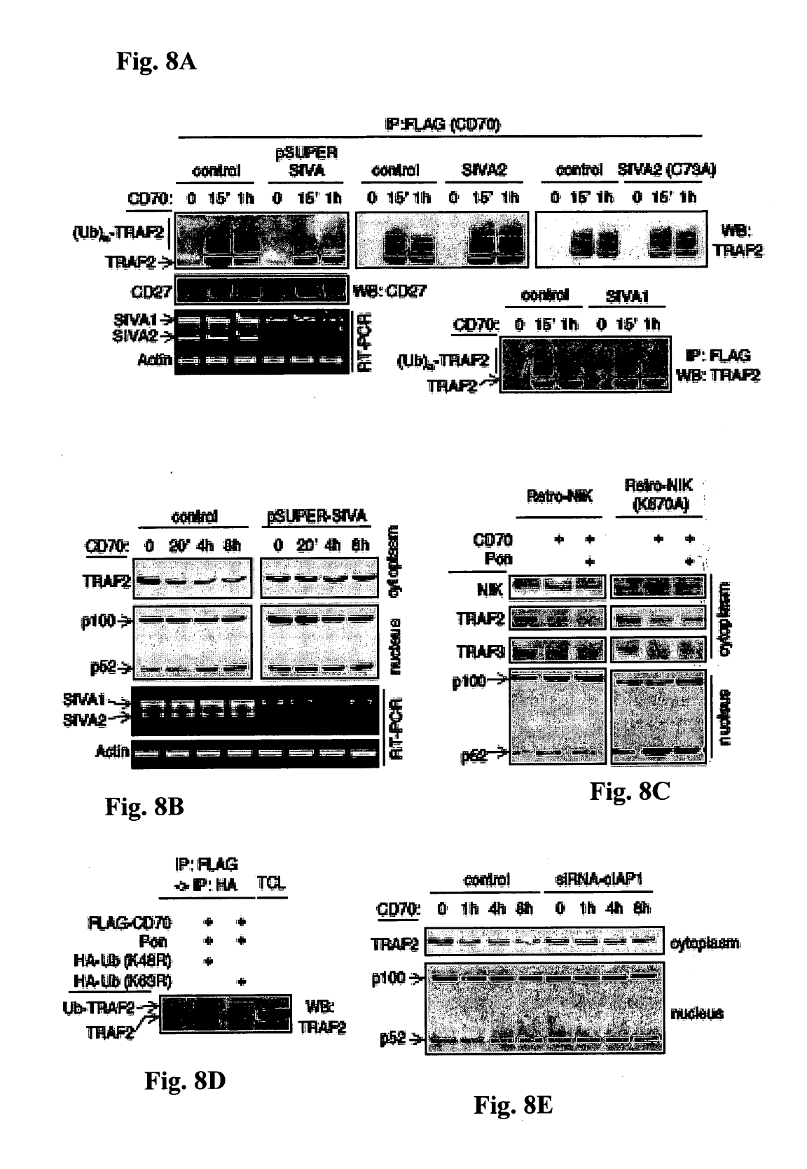

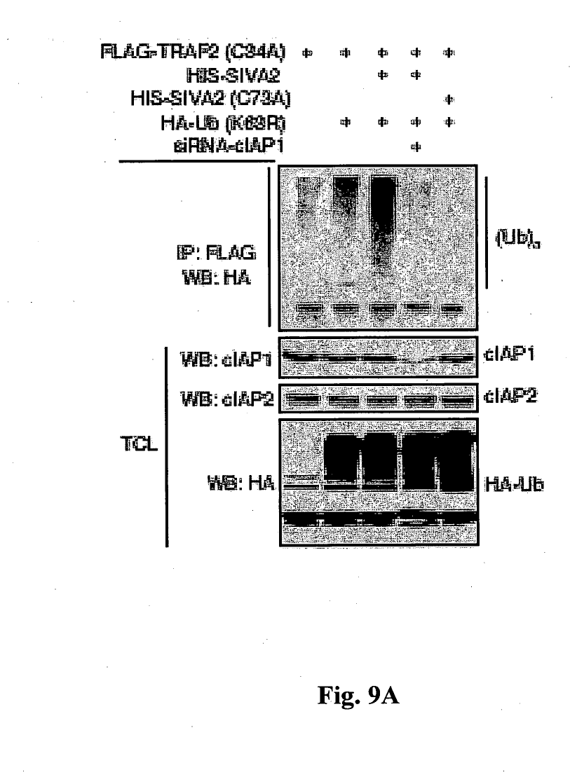

It was previously reported that TRAF2 molecules recruited to CD27 are massively ubiquitinated (Ramakrishnan et al., 2004). To explore the mechanism for the effects of SIVA2 on CD27-induced signaling, it was assessed the impact of SIVA2 on this ubiquitination. As shown in FIG. 8A, knockdown of SIVA attenuated the CD70 ubiquitination of TRAF2 (left panel). In contrast, induction of SIVA2 (middle panel) but not SIVA1 (Right panel), enhanced it.

### Example 9

### SIVA2 Mediates Ubiquitination of Both TRAF2 and cIAP1

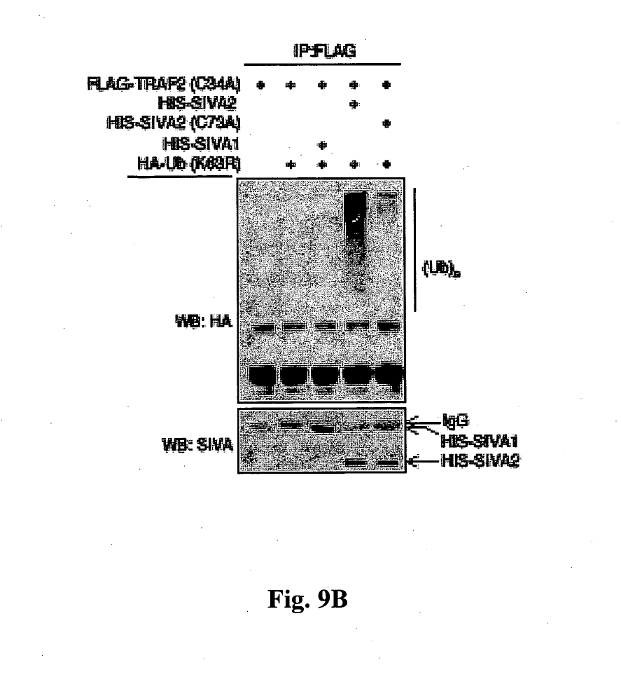

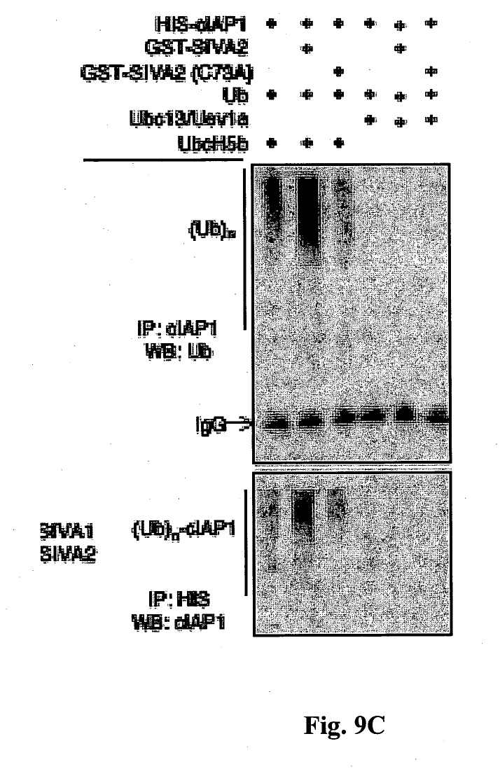

It was previously found that SIVA2 facilitate the self-polyubiquitination of SIVA2 possesses intrinsic ubiquitin-ligase activity, and that it facilitated in-vitro ubiquitination of TRAF2 which was dependent on cysteine residue at position 73 within the CRR in SIVA2, although mutation in this residue does not affect binding of SIVA2 to TRAF2. The effect of mutation in residue 73 of SIVA2 was also demonstrated in transfected cells (FIG. 9A), in which over-expression of wild-type SIVA2, but not SIVA1, markedly increased the K48-linked (though not the K63-linked) polyubiquitination of TRAF2 beyond that observed when TRAF2 was expressed alone, whereas SIVA2 (C73A) hardly affected the ubiquitination (FIG. 9B). Self-ubiquitination of SIVA2 in vitro was not affected by this mutation, but it was drastically reduced by complete deletion of the CRR (not shown).

To verify the physiological significance of this activity of SIVA2, its C73A mutant in cells was expressed in an inducible manner. The mutation was found to ablate the enhancing effect of SIVA2 on the ubiquitination of TRAF2 in the CD27 complex (FIG. 8A, right).

In addition to TRAF2, it was found that cIAP1 was also effectively ubiquitinated by SIVA2, and that this ubiquitination too was compromised by the SIVA2 (C73A) mutation (FIG. 9C). In an attempt to determine the causal relationship between the effects of SIVA2 on cIAP1 and on TRAF2, the effect of cIAP1 knockdown on the SIVA2 effect on TRAF2 was examined. As shown in FIG. 9A, knockdown of cIAP1 dramatically reduced the ubiquitination of TRAF2 in response to SIVA2 expression. Thus, although SIVA2 has the ability to directly ubiquitinate TRAF2 in vitro, its facilitation of TRAF2 ubiquitination within cells is either mediated through enhancement of the ability of cIAP1 to do so, or requires cIAP1 to play a permissive role.

Measurement of the cytoplasmic level of TRAF2 in the tested cells revealed that triggering of CD27 resulted in a significant decrease in the cellular amounts of TRAF2 (FIG. 8B, top left panel), suggesting that its ubiquitination within the receptor complex targets for degradation. The ubiquitin chains whose ligation to TRAF2 was facilitated by SIVA2 were primarily K48-linked (FIG. 8D), as is generally the case with ubiquitination that prompts proteosomal degradation, rising the possibility that this SIVA2 effect contributes to the induction of TRAF2 degradation by CD27. Consistently, knockdown of SIVA expression prevented the downregulation of TRAF2 by CD27 (FIG. 8B, top right panel), whereas induction of SIVA2 enhanced it (FIG. 8C, left panel).

As mentioned above, knockdown of SIVA also resulted in constitutive activation of the alternative NF-κB pathway (FIG. 7C, left panel, and FIG. 8B, right panel). Suppression of cIAP1 expression also results in NE-KB activation (Varfolomeev et al., 2007) (Vince et al., 2007); in addition, it compromises the downregulation of TRAF2 by TNF-RII, another receptor of the TNF/NGF family (Li et al., 2002). As shown in FIG. 8E, knockdown of cIAP1 (like knockdown of SIVA2) compromised the downregulation of TRAF2 by CD27 as well, along with constitutive activation of the alternative NF-κB pathway.

These findings suggested that SIVA2 and cIAP1 play a shared role in the induction of TRAF2 degradation by CD27 and in the regulation of NF-κB.

### REFERENCES

Brummelkamp, T. R., Bernards, R. & Agami, R. A system for stable expression of short interfering RNAs in mammalian cells. Science 296, 550-553 (2002)
Canicio, J., Ruiz-Lozano, P., Carrasco, M., Palacin, M., Chien, K., Zorzano, A., and Kaliman, P. (2001). Nuclear factor kappa B-inducing kinase and Ikappa B kinase-alpha signal skeletal muscle cell differentiation. J Biol Chem 276, 20228-20233.
Cao, C., Ren, X., Kharbanda, S., Koleske, A., Prasad, K. V., and Kufe, D. The ARG tyrosine kinase interacts with Siva-1 in the apoptotic response to oxidative stress. J Biol Chem 276, 11465-11468. 2001
Chu, F., Barkinge, J., Hawkins, S., Gudi, R., Salgia, R., and Kanteti, P. V. Expression of Siva-1 protein or its putative amphipathic helical region enhances cisplatin-induced apoptosis in breast cancer cells: effect of elevated levels of BCL-2. Cancer Res 65, 5301-5309.2005
Chu, F., Borthakur, A., Sun, X., Barkinge, J., Gudi, R., Hawkins, S., and Prasad, K. V. The Siva-1 putative amphipathic helical region (SAH) is sufficient to bind to BCL-XL and sensitize cells to UV. 2004
Collins and Cybulsky, 2001. J Clin Invest. 108:255-64.
Daoud S S, Munson P J, Reinhold W, Young L, Prabhu V V, Yu Q, LaRose J, Kohn K W, Weinstein J N, Pommier Y. Impact of p53 knockout and topotecan treatment on gene expression profiles in human colon carcinoma cells: a pharmacogenomic study. Cancer Res. 2003 Jun. 1; 63(11):2782-93.

Deng, L., Wang, C., Spencer, E., Yang, L., Braun, A., You, J., Slaughter, C., Pickart, C., and Chen, Z. J. Activation of the IkappaB kinase complex by TRAF6 requires a dimeric ubiquitin-conjugating enzyme complex and a unique polyubiquitin chain. Cell 103, 351-361. 98. 2000
Dorsett, Y. and T. Tuschl (2004). “siRNAs: applications in functional genomics and potential as therapeutics.” Nat Rev Drug Discov 3(4): 318-29.
Fanslow, W. C., Clifford, K. N., Seaman, M., Alderson, M. R., Spriggs, M. K., Armitage, R. J., and Ramsdell, F. Recombinant CD40 ligand exerts potent biologic effects on T cells. J Immunol 152, 4262-4269. 1994
Foehr, E. D. et al., 2000. J Biol. Chem. 275:34021-4
Fontanari Krause et al, Abstract 3152, Blood, Vol: 102, 11, Nov. 16, 2003.
Fortin, A., MacLaurin, J. G., Arbour, N., Cregan, S. P., Kushwaha, N., Callaghan, S. M., Park, D. S., Albert, P. R., and Slack, R. S. The proapoptotic gene SIVA is a direct transcriptional target for the tumor suppressors p53 and E2F1. J Biol Chem 279, 28706-28714. 2004
Fred M. Ausubel, R. B., Robert E. Kingston, David D. Moore, J. G. Seidman, John A. Smith, Kevin Struhl, ed. Current protocols in molecular biology. 1996
Glickman, M. H., and Ciechanover, A. The ubiquitin-proteasome proteolytic pathway: destruction for the sake of construction. Physiol Rev 82, 373-428. 2002
Grech, A. P., Amesbury, M., Chan, T., Gardam, S., Basten, A., and Brink, R. TRAF2 differentially regulates the canonical and noncanonical pathways of NF-kappaB activation in mature B cells. Immunity 21, 629-642. 2004
Henke, A., Launhardt, H., Klement, K., Stelzner, A., Zell, R., and Munder, T. Apoptosis in coxsackievirus B3-caused diseases: interaction between the capsid protein VP2 and the proapoptotic protein siva. J Virol 74, 4284-4290. 2000
Hofmann, K., and Falquet, L. A ubiquitin-interacting motif conserved in components of the proteasomal and lysosomal protein degradation systems. Trends Biochem Sci 26, 347-350. 2001
Hofmann, R. M., and Pickart, C. M. In vitro assembly and recognition of Lys-63 polyubiquitin chains. J Biol Chem 276, 27936-27943. 2001
Jacobs S B, Basak S, Murray J I, Pathak N, Attardi L D. Siva is an apoptosis-selective p53 target gene important for neuronal cell death. Cell Death Differ. 2007 July; 14(7):1374-85.
Kalkum, M., Lyon, G. J. & Chait, B. T. Detection of secreted peptides by using hypothesis-driven multistage mass spectrometry. Proc Natl Acad Sci USA 100, 2795-2800 (2003).
Kajiura, F., Sun, S., Nomura, T., Izumi, K., Ueno, T., Bando, Y., Kuroda, N., Han, H., Li, Y., Matsushima, A., et al. (2004). NF-kappa B-inducing kinase establishes self-tolerance in a thymic stroma-dependent manner. J Immunol 172, 2067-2075.
Karin, M., and Ben-Neriah, Y. Phosphorylation meets ubiquitination: the control of NF-[kappa]B activity. Annu Rev Immunol 18, 621-663. 2000
Kim, D. H., M. A. Behlke, et al. (2005). “Synthetic dsRNA Dicer substrates enhance RNAi potency and efficacy.” Nat Biotechnol 23(2): 222-6.
Kovalenko, A., Chable-Bessia, C., Cantarella, G., Israel, A., Wallach, D., and Courtois, G. The tumour suppressor CYLD negatively regulates NF-kappaB signalling by deubiquitination. Nature 424, 801-805. 2003
Lee, Z. H., Lee, S. E., Kwack, K., Yeo, W., Lee, T. H., Bae, S. S., Suh, P. G., and Kim, H. H. Caspase-mediated cleavage of TRAF3 in FasL-stimulated Jurkat-T cells. J Leukoc Biol 69, 490-496. 2001
Leonard, W. J. et al., 1995. Immunol Rev. 148:97-114.
Liao, G., Zhang, M., Harhaj, E. W., and Sun, S. C. Regulation of the NF-kappaB-inducing kinase by tumor necrosis factor receptor-associated factor 3-induced degradation. J Biol Chem 279, 26243-26250. 2004.
Li, X., Yang, Y. and Ashwell, J. D. (2002) TNF-RII and c-IAP1 mediate ubiquitination and degradation of TRAF2. Nature, 416, 345-347.
Lin, X. et al., 1999. Immunity 10:271-80.
Ling, L. et al., 1998. Proc Natl Acad Sci USA. 95:3792-7
Lois, C., Hong, E. J., Pease, S., Brown, E. J., and Baltimore, D. Germline transmission and tissue-specific expression of transgenes delivered by lentiviral vectors. Science 295, 868-872. 2002.
Malinin, N. L., Boldin, M. P., Kovalenko, A. V., and Wallach, D. MAP3K-related kinase involved in NF-kappaB induction by TNF, CD95 and IL-1. Nature 385, 540-544. 1997.
Matsushima, A. et al., 2001. J Exp Med. 193:631-6
Matsumoto, M. et al., 1999. J Immunol. 163:1584-91. Mattson and Camandola, 2001. J Clin Invest. 107:247-54.
Mercurio F. and Manning A. M., 1999. Curr Opin Cell Biol. 11:226-32.
Miyawaki, S., Nakamura, Y., Suzuka, H., Koba, M., Yasumizu, R., Ikehara, S., and Shibata, Y. (1994). A new mutation, aly, that induces a generalized lack of lymph nodes accompanied by immunodeficiency in mice. Eur J Immunol 24, 429-434.
Montigny, C., et al. Fe2+-catalyzed oxidative cleavages of Ca2+-ATPase reveal novel features of its pumping mechanism. J Biol Chem 279, 43971-43981 (2004).
Nocentini, G., and Riccardi, C. GITR: a multifaceted regulator of immunity belonging to the tumor necrosis factor receptor superfamily. Eur J Immunol 35, 1016-1022. 2005
Padanilam, B. J., Lewington, A. J., and Hammerman, M. R. Expression of CD27 and ischemialreperfusion-induced expression of its ligand Siva in rat kidneys. Kidney Int 54, 1967-1975. 1998
Petit P X, P. B., Mrugala D, Biard-Piechaczyk M, Benichou S. SIVA: A new intracellular ligand of the CD4 receptor modulating T lymphocyte apoptosis via a caspase-dependent mitochondrial pathway, Paper presented at: ISAC congress XXII (France: WILEY-LISS, DIV JOHN WILEY & SONS INC, 111 RIVER ST, HOBOKEN, N.J. 07030 USA). 2004
Pickart, C. M. Mechanisms underlying ubiquitination. Annu Rev Biochem 70, 503-533. 2001
Pomerantz, J. L., and Baltimore, D. (2002). Two pathways to NF-kappaB. Mol Cell 10, 693-695.
Prasad, K. V., Ao, Z., Yoon, Y., Wu, M. X., Rizk, M., Jacquot, S., and Schlossman, S. F. CD27, a member of the tumor necrosis factor receptor family, induces apoptosis and binds to Siva, a proapoptotic protein. Proc Natl Acad Sci USA 94, 6346-6351. 1997
Prasad, K. V., Ao, Z., Yoon, Y., Wu, M. X., Rizk, M., Jacquot, S., and Schlossman, S. F. (1997). CD27, a member of the tumor necrosis factor receptor family, induces apoptosis and binds to Siva, a proapoptotic protein. Proc Natl Acad Sci USA 94, 6346-6351.
Py, B., Slomianny, C., Auberger, P., Petit, P. X., and Benichou, S. Siva-1 and an alternative splice form lacking the death domain, Siva-2, similarly induce apoptosis in T lymphocytes via a caspasedependent mitochondrial pathway. J Immunol 172, 4008-4017. 2004
Qin, L. F., Lee, T. K., and Ng, I. O. Gene expression profiling by cDNA array in human hepatoma cell line in response to cisplatin treatment. Life Sci 70, 1677-1690. 2002
Ramakrishnan, P., Wang, W., and Wallach, D. Receptor-specific signaling for both the alternative and the canonical NF-kappaB activation pathways by NF-kappaB-inducing kinase. Immunity 21, 477-489. 2004
Regnier, C. H. et al., 1997. Cell 90:373-83.
Rigaut, G., Shevchenko, A., Rutz, B., Wilm, M., Mann, M., and Seraphin, B. A generic protein purification method for protein complex characterization and proteome exploration. Nat Biotechnol 17, 1030-1032. 1999
Schreiber, E., Matthias, P., Muller, M. M., and Schaffner, W. Rapid detection of octamer binding proteins with ‘mini-extracts’, prepared from a small number of cells. Nucleic Acids Res 17, 6419. 1989
Senftleben, U., Cao, Y., Xiao, G., Greten, F. R., Krahn, G., Bonizzi, G., Chen, Y., Hu, Y., Fong, A., Sun, S. C., and Karin, M. Activation by IKKalpha of a second, evolutionary conserved, NF-kappa B signaling pathway. Science 293, 1495-1499. 2001
Shinkura, R., Kitada, K., Matsuda, F., Tashiro, K., Ikuta, K., Suzuki, M., Kogishi, K., Serikawa, T., and Honjo, T. Alymphoplasia is caused by a point mutation in the mouse gene encoding Nf-kappa binducing kinase. Nat Genet. 22, 74-77. 107. 1999
Spinicelli, S., Nocentini, G., Ronchetti, S., Krausz, L. T., Bianchini, R., and Riccardi, C. GITR interacts with the pro-apoptotic protein Siva and induces apoptosis. Cell Death Differ 9, 1382-1384. 2002
Sylla, B. S. et al., 1998. Proc Natl Acad Sci USA. 95:10106-11
Soutschek, J., A. Akinc, et al. (2004). “Therapeutic silencing of an endogenous gene by systemic administration of modified siRNAs.” Nature 432(7014): 173-8.
Spinicelli, S., Nocentini, G., Ronchetti, S., Krausz, L. T., Bianchini, R., and Riccardi, C. GITR interacts with the pro-apoptotic protein Siva and induces apoptosis. Cell Death Differ 9, 1382-1384. 2002
Slawson et al., Journal of cellular Biochemistry 97:71-83, 2006.
Varfolomeev, E., Blankenship, J. W., Wayson, S. M., Fedorova, A. V., Kayagaki, N., Garg, P., Zobel, K., Dynek, J. N., Elliott, L. O., Wallweber, H. J., Flygare, J. A., Fairbrother, W. J., Deshayes, K., Dixit, V. M. and Vucic, D. (2007) IAP Antagonists Induce Autoubiquitination of c-IAPs, NF-kappaB Activation, and TNFalpha-Dependent Apoptosis. Cell, 131, 669-681.
Vince, J. E., Wong, W. W., Khan, N., Feltham, R., Chau, D., Ahmed, A. U., Benetatos, C. A., Chunduru, S. K., Condon, S. M., McKinlay, M., Brink, R., Leverkus, M., Tergaonkar, V., Schneider, P., Callus, B. A., Koentgen, F., Vaux, D. L. and Silke, J. (2007) IAP Antagonists Target cIAP1 to Induce TNFalpha-Dependent Apoptosis. Cell, 131, 682-693.
Wajant, H., and Scheurich, P. (2004). Analogies between Drosophila and mammalian TRAF pathways. Prog Mol Subcell Biol 34, 47-72.
Whelan S A, Hart G W. Identification of O-GlcNAc sites on proteins. Methods Enzymol. 2006; 415:113-33.
Xiao, G., Fong, A., and Sun, S. C. Induction of p100 processing by NF-kappaB-inducing kinase involves docking IkappaB kinase alpha (IKKalpha) to p100 and IKKalpha-mediated phosphorylation. J Biol Chem 279, 30099-30105. 2004
Xiao, G., and Sun, S. C. Negative regulation of the nuclear factor kappa B-inducing kinase by a cis-acting domain. J Biol Chem 275, 21081-21085. 2000
Xu, L. G., Li, L. Y., and Shu, H. B. (2004). TRAF7 potentiates MEKK3-induced AP1 and CHOP activation and induces apoptosis. J Biol Chem 279, 17278-17282.
Xue, L., Chu, F., Cheng, Y., Sun, X., Borthakur, A., Ramarao, M., Pandey, P., Wu, M., Schlossman, S. F., and Prasad, K. V. Siva-1 binds to and inhibits BCL-X(L)-mediated protection against UV. 2002
Yamamoto and Gaynor, 2001. J Clin Invest. 107:135-142
Yoon, Y., Ao, Z., Cheng, Y., Schlossman, S. F., and Prasad, K. V. Murine Siva-1 and Siva-2, alternate splice forms of the mouse Siva gene, both bind to CD27 but differentially transduce apoptosis. Oncogene 18, 7174-7179. 1999

1. A stability-improved SIVA2 or salt thereof comprising one or more post translation modification(s) selected from the group consisting of (i) O-GlcNAcylation; (ii) phosphorylation at serine residues 5, 50, and 51 and (iii) ubiquitination on residues, K17 and/or K99; and (iv) a combination of (i) to (iii).

2. The stability-improved SIVA2 according to claim 1 (ii), wherein SIVA2 is also phosphorylated at serine residues 21, 26, and 35.

3. A method of preparing a stability-improved SIVA2 comprising one or more post translation modification(s) selected from the group consisting of (i) O-GlcNAcylation; (ii) phosphorylation at serine residues 5, 50, 51 of SIVA2; (iii) ubiquitination on SIVA2 residues, K17 and/or K99; and (iv) a combination of (i) to (iii), the method comprising over-expressing in an eukaryotic cell recombinant or endogenous SIVA2 and increasing in said eukaryotic cell the levels of (a) TRAF2, (b) a ring-finger mutant of cl API, (c) a O-GlcNAc transferase, (d) an inhibitor of O-GlcNAcase, (e) UDP-GlcNac, (f) a combination of (a) to (e) or (g) NF-κB-inducing kinase (NIK) and any one of (a) to (f).

4. The method according to claim 3, wherein the method is carried out ex-vivo, and further comprises culturing said cell under conditions allowing production of said stability-improved SIVA2 and recovering the resulting SIVA2 from the culture.

5. A stability-improved SIVA2 prepared according to the method of claim 3 or 4.

6. A host cell comprising a stability-improved SIVA2 comprising one or more of the post translation modification(s) selected from the group consisting of (i) O-GlcNAcylation; (ii) phosphorylation at serine residues 5, 50, and 51; (iii) ubiquitination on residues, K17 and/or K99; and (iv) a combination of (i) to (iii).

7. A pharmaceutical composition comprising a pharmaceutically acceptable carrier and a stability-improved SIVA2 or salt thereof comprising one or more post translation modification(s) selected from the group consisting of (i) O-GlcNAcylation; (ii) phosphorylation at serine residues 5, 50, and 51; (iii) ubiquitination on residues, K17 and/or K99; and (iv) a combination of (i) to (iii).

8.-11. (canceled)

12. A method for stabilizing SIVA2 comprising contacting SIVA2 with an O-GlcNac transferase, TRAF2, an inhibitor of O-GlcNAcase, an inhibitor CIAP1 activity, a ring-finger mutant of cIAP1 such as H588A or a combination thereof, wherein said contacting is carried out in vivo, in vitro or ex-vivo.

13.-19. (canceled)

20. A complex of SIVA2 or stability-improved SIVA2 with cIAP.

21. A method for screening a molecule capable of modulating signaling by a member of the TNF/NGF receptor family in a disease disorder or condition comprising contacting SIVA2 with cIAP and/or TRAF2, monitoring the level of the complex of SIVA2 with cIAP and/or TRAF2 in the presence and in the absence of a candidate molecule, wherein a change in the level of SIVA2-cIAP and/or SIVA2-TRAF2 complex in the presence of the candidate molecule is indicative that the candidate molecule modulates signaling by said member of the TNF/NGF family.

22. The method according to claim 21, wherein the method is for screening a molecule capable of downregulating signaling by the member of the TNF/NGF receptor family in a disease disorder or condition and wherein the candidate molecule increases the level of the complex.

23. The method according to claim 22, wherein the disease, disorder or condition is an autoimmune disease, disorder or condition or in kidney ischemia.

24. The method according to claim 21, wherein the method is for screening a molecule capable of prolonging signaling by the member the TNF/NGF receptor family in a disease, disorder or condition and wherein the candidate molecule decreases the level of the complex.

25. The method according to claim 24, wherein the disease, disorder or condition is associated with immunosuppression.

26. A method for screening a molecule capable of modulating signaling by a member of the TNF/NGF receptor family in a disease, disorder or condition comprising inducing SIVA2 stability in the presence and in the absence of a candidate molecule, wherein a change in the level of stability-induced SIVA2 in the presence of a candidate molecule is indicative that the candidate molecule can modulate signaling by the member of the TNF/NGF receptor family.

27. The method according to claim 26, wherein the method is for screening a molecule capable of downregulating signaling by the member of the TNF/NGF receptor family in a disease, disorder or condition and wherein the candidate molecule increases the level of stabilized SIVA2.

28. The method according to claim 27, wherein the disease, disorder or condition is an autoimmune disease, disorder or condition or in kidney ischemia.

29. The method according to claim 26, wherein the method is for screening a molecule for capable of prolonging signaling by the member of the TNF/NGF receptor family in a disease, disorder or condition and wherein the candidate molecule decreases the level of stabilized SIVA2.

30. The method according to claim 29, wherein the disease, disorder or condition is associated with immunosuppression.

31. A method for treating a disease, disorder, or condition in which a signaling pathway by a member of the TNF/NGF receptor family is associated with the pathogenesis or course of the disease, disorder, or condition wherein the method comprises administration of a therapeutically effective amount an agent capable of altering SIVA2 stability selected from (i) an agent capable of modulating O-GlcNacidation, (ii) an agent capable of modulating TRAF2 activity, (iii) an agent capable of modulating CIAP1 activity, and (iv) a ring-finger mutant of cIAP1.

32. The method according to claim 31, wherein altering SIVA2 stability consists on improving SIVA2 stability.

33. The method according to claim 31 or 32, wherein the agent is selected form O-GlcNac transferase, TRAF2, an inhibitor of O-GlcNAcase, an inhibitor CIAP1 activity, a ring-finger mutant of cIAP1 such as H588A or a combination thereof.

34. The method according to claim 33, for treating cancer, an inflammatory disease, and/or an autoimmune disease.

35. The method according to claim 31, wherein altering SIVA2 stability consists on diminishing SIVA2 stability.

36. The method according to claim 31 or 35, wherein the agent is an inhibitor of O-GlcNac transferase, inhibitor of TRAF2, an O-GlcNAcase, CIAP1, or a combination thereof.

37. The method according to claim 36, for treating an immune deficiency or ischemia/reperfusion.

38. The method of claim 31, wherein the ring-finger mutant of cIAP1 is H588A.


SEQUENCE LISTING (11 sequences)

SEQ ID NO: 1 - DNA, 9 bp, Homo sapiens
ttcaagaga

SEQ ID NO: 2 - DNA, 64 bp, Homo sapiens
gatcccctgaataaacctctttatatttcaagagaatataaagaggtttattcatttttggaaa

SEQ ID NO: 3 - DNA, 64 bp, Homo sapiens
agcttttccaaaaatgaataaacctctttatattctcttgaaatataaagaggtttattcaggg

SEQ ID NO: 4 - DNA, 64 bp, Homo sapiens
gatccccactgcagtgacatgtacgattcaagagatcgtacatgtcactgcagttttttggaaa

SEQ ID NO: 5 - DNA, 64 bp, Homo sapiens
agcttttccaaaaaactgcagtgacatgtacgatctcttgaatcgtacatgtcactgcagtggg

SEQ ID NO: 6 - DNA, 64 bp, Homo sapiens
gatccccgctacctgttccatggccattcaagagatggccatggaacaggtagctttttggaaa

SEQ ID NO: 7 - DNA, 64 bp, Homo sapiens
agcttttccaaaaagctacctgttccatggccatctcttgaatggccatggaacaggtagcggg

SEQ ID NO: 8 - DNA, 33 bp, Artificial Sequence
  Description of Artificial Sequence Synthetic oligonucleotide
cgcggatccaacatgcccaagcggagctgcccc

SEQ ID NO: 9 - DNA, 33 bp, Artificial Sequence
  Description of Artificial Sequence Synthetic oligonucleotide
ccgctcgaggccagcctcaggtctcgaacatgg

SEQ ID NO: 10 - DNA, 528 bp, Homo sapiens
atgcccaagcggagctgccccttcgcggacgtggccccgctacagctcaaggtccgcgtgagccagagggagttgagccgcggcgtgtgcgccgagcgctactcgcaggaggtcttcgagaagaccaagcgactcctgttcctcggggcccaggcctacctggaccacgtgtgggatgaaggctgtgccgtcgttcacctgccagagtccccaaagcctggccctacaggggccccgagggctgcacgtgggcagatgctgattggaccagacggccgcctgatcaggagccttgggcaggcctccgaagctgacccatctggggtagcgtccattgcctgttcctcatgcgtgcgagccgtggatgggaaggcggtctgcggtcagtgtgagcgagccctgtgcgggcagtgtgtgcgcacctgctggggctgcggctccgtggcctgtaccctgtgtggcctcgtggactgcagtgacatgtacgagaaagtgctgtgcaccagctgtgccatgttcgagacctga

SEQ ID NO: 11 - DNA, 343 bp, Homo sapiens
atgcccaagcggagctgccccttcgcggacgtggccccgctacagctcaaggtccgcgtgagccagagggagttgagccgcggcgtgtgcgccgagcgctactcgcaggaggtcttcgacccatctggggtagcgtccattgcctgttcctcatgcgtgcgagccgtggatgggaaggcggtctgcggtcagtgtgagcgagccctgtgcgggcagtgtgtgcgcacctgctggggctgcggctccgtggcctgtaccctgtgtggcctcgtggactgcagtgacatgtacgagaaagtgctgtgcaccagctgtgccatgttcgagacctgaggctggctca